(19) United States
(12) Patent Application Publication
(10) Pub. No.: US 2023/0183333 A1
(43) Pub. Date: Jun. 15, 2023

(54) COMPOSITION FOR THE TREATMENT OF PHILADELPHIA CHROMOSOME-POSITIVE ACUTE LYMPHOBLASTIC LEUKEMIA

(71) Applicants: Hassan JUMAA-WEINACHT, Blaubeuren (DE); Mouhannad JUMAA, San Jose, CA (US)

(72) Inventors: Hassan JUMAA-WEINACHT, Blaubeuren (DE); Mouhannad JUMAA, San Jose, CA (US)

(21) Appl. No.: 17/999,330

(22) PCT Filed: May 18, 2021
(86) PCT No.: PCT/US2021/033034

§ 371 Date: Nov. 18, 2022

Related U.S. Application Data
(60) Provisional application No. 63/026,522, filed on May 18, 2020.

(51) Int. Cl. C07K 16/24; A61P 35/02; A61K 39/395; A61K 31/506; A61K 31/519

(52) U.S. Cl. CPC C07K 16/244; A61P 35/02; A61K 39/3955; A61K 31/506; A61K 31/519; A61K 2039/505

(57) ABSTRACT

The present invention provides compositions comprising antibodies to interleukin 7 receptor (IL7R), as well methods for treatment using the IL7R antibodies.

### CROSS REFERENCE TO RELATED APPLICATIONS

The application claims priority benefit from U.S. Provisional Patent Application 63/026,522, filed May 18, 2020 which is incorporated by reference herein in its entirety.

### BACKGROUND OF THE INVENTION

Acute lymphoblastic leukemia (ALL) is the most common blood cancer and affects white blood cells, particularly lymphocytes. One subtype of this cancer is Philadelphia chromosome positive acute lymphoblastic leukemia (Ph+ALL). While less common in children, about 30% of ALL in adults are positive for the Philadelphia chromosome (Ph+). The result of this Ph+ is that these cancers express an oncogenic fusion kinase termed BCR-ABL that drives the malignant transformation of precursor B cells.

Tyrosine kinase inhibitors (TKIs), such as ABL kinase inhibitors, are used to treat patients with Ph+ALL. TKIs such as imatinib are used in combination with chemotherapy and this treatment is the current standard of care for treatment of Ph+ALL. However, the development and/or presence of kinase inhibitor-resistant clones results in relapse and often an incurable outbreak of the leukemia disease.

### BRIEF SUMMARY OF THE INVENTION

The present invention provides a composition comprising an antibody that specifically binds to interleukin 7 receptor (IL7R), wherein the antibody does not prevent binding of IL7R to interleukin 7 (IL7).

In some embodiments, the antibody does not bind to a ligand binding site of IL7R.

In some embodiments, the antibody binds to a ligand binding site of IL7R.

The present invention also provides a composition comprising an antibody that specifically binds to interleukin 7 receptor (IL7R), wherein the antibody comprises:

a variable heavy domain (VH) comprising vhCDRs 1-3 having amino acid sequences of SEQ ID NOs:2-4, respectively; and

a variable light domain (VL) comprising vlCDRs 1-3 having amino acid sequences of SEQ ID NOs:9-11, respectively.

The present invention also provides a composition comprising an antibody that specifically binds to interleukin 7 receptor (IL7R), wherein the antibody comprises:

a variable heavy domain (VH) from a heavy chain having an amino acid sequence of SEQ ID NO:1; and

a variable light domain (VL) from a light chain having an amino acid sequence of SEQ ID NO:8.

In some embodiments, the antibody comprises:

a heavy chain having an amino acid sequence of SEQ ID NO:1; and

a light chain having an amino acid sequence of SEQ ID NO:8.

The present invention also provides a composition comprising an antibody that specifically binds to interleukin 7 receptor (IL7R), wherein the antibody comprises:

a variable heavy domain (VH) comprising vhCDRs 1-3 having amino acid sequences of SEQ ID NOs:2, 6, and 4, respectively;

a variable light domain (VL) comprising vlCDRs 1-3 having amino acid sequences of SEQ ID NOs:9-11, respectively.

The present invention also provides a composition comprising an antibody that specifically binds to interleukin 7 receptor (IL7R), wherein the antibody comprises:

a variable heavy domain (VH) from a heavy chain having an amino acid sequence of SEQ ID NO:5;

a variable light domain (VL) from a light chain having an amino acid sequence of SEQ ID NO:8.

In some embodiments, composition of claim 8, wherein the antibody comprises:

a heavy chain having an amino acid sequence of SEQ ID NO:5; and

a light chain having an amino acid sequence of SEQ ID NO:8.

The present invention also provides a composition comprising an antibody that specifically binds to interleukin 7 receptor (IL7R), wherein the antibody comprises:

a variable heavy domain (VH) from a heavy chain having an amino acid sequence of SEQ ID NO:7;

a variable light domain (VL) from a light chain having an amino acid sequence of SEQ ID NO:8.

In some embodiments, the antibody comprises:

a heavy chain having an amino acid sequence of SEQ ID NO:7; and

a light chain having an amino acid sequence of SEQ ID NO:8.

The present invention provides a composition comprising an antibody that competes with the antibody of any one of claims 1 to 11 for binding to interleukin 7 receptor (IL7R).

The present invention provides a composition comprising:

a first nucleic acid comprising a first polynucleotide sequence encoding the variable heavy domain (VH) of the antibody of any one of claims 1 to 12, and

a second nucleic acid comprising a second polynucleotide sequence encoding the variable light domain (VL) of the same antibody.

In some embodiments, the invention provides an expression vector comprising the first and second nucleic acids as described herein.

In some embodiments, the invention provides a host cell comprising the expression vector as described herein.

The present invention provides a method of making the antibody as described herein comprises:

a) culturing the host cell of claim 14 under conditions wherein the antibody is produced; and

b) recovering the antibody.

In some embodiments, the method of making the antibody further comprises humanizing the antibody.

The present invention provides a method of treating acute leukemia in a patient in need comprising administering an antibody that specifically binds to interleukin 7 receptor (IL7R) to the patient.

In some embodiments, the antibody that specifically binds to interleukin 7 receptor (IL7R) is the antibody as described herein.

In some embodiments, the antibody is used as a monotherapy.

In some embodiments, the antibody is used in combination with a standard chemotherapy.

In some embodiments, the antibody is administered prior to, simultaneously with, or subsequent to the administration of one or more chemotherapeutic agents.

In some embodiments, the antibody is used in combination with a kinase inhibitor (such as imatinib).

In some embodiments, the antibody is administered prior to, simultaneously with, or subsequent to the administration of the kinase inhibitor (such as imatinib).

In some embodiments, prior to the treatment the patient has relapsed following TKI therapy.

In some embodiments, the patient has circulating malignant cells.

In some embodiments, the circulating malignant cells in the patient expresses IL7R.

In some embodiments, the leukemia is resistant leukemia.

In some embodiments, the leukemia is acute lymphoblastic leukemia (ALL).

In some embodiments, the ALL is Philadelphia chromosome positive acute lymphoblastic leukemia (Ph+ALL).

In some embodiments, the ALL is resistant ALL.

In some embodiments, the ALL is pediatric ALL.

The present invention also provides for the use of an antibody as described herein for treating leukemia (including resistant leukemia).

The present invention also provides for the use of an antibody as described herein for treating ALL.

The present invention also provides for the use of an antibody as described herein for treating Philadelphia chromosome positive acute lymphoblastic leukemia (Ph+ALL).

The present invention also provides for the use of an antibody as described herein for treating resistant ALL.

The present invention also provides for the use of an antibody as described herein for treating pediatric ALL.

A method of treating a patient with Ph+ALL by administering an anti-IL7R antibody that does not compete for binding with IL7.

### BRIEF DESCRIPTION OF THE DRAWINGS

FIG. 1: Gene expression profiles of BCR-ABL1-transformed cells. Bone marrow (BM)-derived pre-B cells isolated from two wild type (WT) mice were used to generate 6 independent BCR-ABL1 transformed cell lines or 6 control cell lines expressing empty vector (EV). For expression profiling, RNA was isolated using ReliaPrepTM RNA Miniprep System (MM). All samples were subjected to RNA quality control test before the RNA-seq was applied. (a) Heatmap representation of selected genes related to cytokine receptor signaling from the previously specified GOs (see below) in BCR-ABL1-transformed cells compared to the EV-transduced cells. Samples are represented in columns while rows show genes. An average linking method based on Pearson correlation distance metric was applied to duster rows and columns. (b) Gene Set Enrichment Analysis (GSEA) showing upregulation of gene set belonging to the JAK-STAT signaling pathway in BCR-ABL1 group. Heatmap representation (left) of the top 28 deregulated genes (core enrichment genes) in BCR-ABL1 versus EV-transduced samples (Blue: down-regulated; Red: up-regulated; NES=Normalized Enrichment Score; FDR=False Discovery Rate). A two-sided signal-to-noise metric was used to rank the genes. For a calculated GSEA nominal p-values of 0, we present them as p<0.001 (otherwise, exact p-values are shown). Multiple hypothesis testing correction is represented by the estimated FDR.

FIGS. 2A, 2B, 2C, 2D and 2E: Regulation of IL7R and CXCR4 expression levels in BCR-ABL+ ALL. FIG. 2(a) depicts the correlation analysis between IL7R and CXCR4 expression levels in 68 pediatric BCR-ABL⁺ ALL patients. Two-tailed Sperman correlation analysis; exact p value=0.000000011054191. (2b) Flow cytometry analysis showing that imatinib treatment (1μM;15 hours) leads to increased expression of IL7R and CXCR4 on the cell surface of BCR-ABL-transformed cells. The results are representative of 3 independent experiments. (2c) Quantitative RT-PCR showing the IL7R and CXCR4 and other associated factors are regulated at the level of transcription. N=3 independent samples per group, and error bars represent mean±SD. Unpaired t-test, two-sided, p1 exact value=0.000035569652955. (2d) BCR-ABL1-transformed WT pre-B cells were treated with 1 μM imatinib and with different concentrations of IL7 as indicated for 6 days. N=3 independent samples per group, and error bars represent mean±SD. One-way ANAOVA. Dunnett's multiple comparisons test was performed to day 6, compared to control group (Vehicle). Adjusted p values: Vehicle vs. 1.25 ng/ml IL7 ***p=0.0003 , Vehicle vs. 2.5 ng/ml IL7 ***p=0.0002, Vehicle vs. 5 ng/ml IL7 ***p=0.0002. (2e) Treatment of BCR-ABL1 transformed cells with imatinib leads to cell death and concomitant incubation with IL7, but not CXCL12, reverses this effect. N=3 independent samples per group, and error bars represent mean±SD. Unpaired t-test, two-sided, ****p1 exact value=0.000003416387742, ****p2 exact value=0.000002948485097, ****p3 exact value=0.000000000342967.

FIGS. 3A to 3G: IL7R is required for BCP-ALL leukemogenesis: (3a) Bone marrow-derived pre-B cells from WT mice were transduced with either an empty vector (EV) or with BCR-ABL1. Viability of the cells after IL7 withdrawal was determined by flow cytometry. The results are representative of 3 independent experiments. (3b)The enrichment of CD19+GFP+ cells in relative to control after IL7 withdrawal (n=7 per group, and error bars represent mean±SD. Unpaired t-test, two-sided, exact p value ****p<0.000000000000001). (c3-3g) IL7R^{fl/fl}pre-B cells were transformed with BCR-ABL1 and were then transduced with either ER^{T2} or Cre-ER^{T2}. Cells were treated with either tamoxifen (Tam) to induce Cre expression, or with ethanol (Et). (3c) PCR analysis for IL7R deletion (IL7R^{Δ}), SRp20 was used as a loading control. Cells transduced with ER^{T2} were used as control. The results are representative of 3 independent experiments. bp: base pair. (3d) Extracellular staining for IL7R protein in IL7R^{fl/fl}pre-B cells after 72 hours of Cre-induction. The results are representative of 3 independent experiments. (3e) The percentage of living cells were determined by flow cytometry using Sytox as an excluding dead cell stain. N=3 independent samples per group, and error bars represent mean±SD. One-way ANAOVA. Dunnett's multiple comparisons test was performed to day 5, compared to control group (ER^{T2} Et). Adjusted p values: ER^{T2} Et vs. ER^{T2} Tam n.s. p=0.1769, ER^{T2} Et vs. Cre-ER^{T2} Et n.s. p=0.6325, ER^{T2} Et vs. Cre-ER^{T2} Tam ****p<0.0001. (3f) Luciferase bioimaging and (3g) survival curves of NOD-SCID mice that were injected with 1×10⁶ IL7R^{fl/fl}-BCR-ABL1 pre-B cells containing either a Cre-ER^{T2} construct or an ER^{T2}. (n=21 per group). Mantel-Cox-log-rank test, ***p value=0.0002.

FIGS. 4A to 4E: Synergism between CXCR4 and IL7R in BCR-ABL1 induced cells. (4a) BCR-ABL1 acts downstream of CXCR4. Shown are flow cytometry plots for Ca²⁺ mobilization in response to CXCL12 treatment (100 ng/ml) in WT as compared to BCR-ABL1 transformed cells with or without kinase inhibitor treatment as indicated. The results are representative of 3 independent experiments. (4b) Proximity ligation analysis (PLA) to detect the dose association of IL7R and CXCR4 on the surface of WT BM-derived pre-B cells as compared with mature B cells (which lacks IL7R expression; see FIG. 14a). (4c) PLAs showing increased association of IL7R and CXCR4 on the surface of BCR-ABL1 transformed cells. (4d) PLA showing recruitment of Jak3 to CXCR4 in BCR-ABL1 transformed cells. This association was lost upon inducible deletion of IL7R using Cre-ER^{T2} (IL7R^{Δ}) system. Close proximity is represented by red dots. (4b-4d) Quantification shows number of signals per cell, error bars represent mean±SD. Unpaired t-test, two-sided. The results shown are representative of 3 independent experiments. Exact p values (4c) 0.00000000006167, approximate p value (4b) <0,000000000000001, approximate p value (4d) <0,000000000000001. (4e) Left panel, western blot analysis for increased phosphorylation of the Jak kinases by BCR-ABL1 activity. Right panel, western blot showing that inducible deletion of CXCR4 or IL7R results in reduced phosphorylation of all Jak kinases. The results are representative of 3 independent experiments. KDa: Kilo Dalton.

FIGS. 5A to 5E: Negative feedback regulating BCR-ABL1-induced transformation. (5a) Western blot analysis for increased phosphorylation of STAT5 and FoxO1 by BCR-ABL1 activity. (5b) Western blot analysis showing that constitutively active STAT5 (STAT5-CA) enhances FoxO1 phosphorylation. The western blot results in a and b are representative of 3 independent experiments; KDa: Kilo Dalton. (5c) Ectopic expression of STAT5-CA in WT pre-B cells (left) or BCR-ABL1-transformed cells (right) leads to downregulation of surface expression of IL7R protein after two days of transduction. (5d) Reverse transcriptase PCR analysis showing downregulation of IL7R due to constitutive activity of STAT5; actin was used as a loading control. cDNA was prepared from sorted cells after two days of transduction. bp: base pair. (5e) Loss of BCR-ABL1 transformed pre-B cells which overexpress constitutively active STAT5 (STAT5-CA). Representative flow cytometry plots at day 2 and 7 after transduction. EV: Empty vector. (5a-e) The results are representative of 3 independent experiments.

FIGS. 6A to 6F show FoxO1 is required for BCR-ABL1 driven leukemogenesis. FoxO1^{fl/fl} pre-B cells were transformed with BCR-ABL1 and were then transduced with Cre-ER^{T2}. Cells were treated with either tamoxifen (Tam) to induce Cre expression, or with ethanol (Et). (6a) PCR analysis for Fox O1 deletion (FoxO1^{Δ}), SRp20 was used as a loading control. Cells transduced with ER^{T2} were used as control. bp: base pair. (6b) The percentage of living cells were determined by flow cytometry using Syotx as excluding dead cell stain. (6c) The fold change of living cells after treatment with either ethanol or tamoxifen at different time points. N=3 independent samples per group, and error bars represent mean±SD. One-way ANAOVA. Dunnett's multiple comparisons test was performed to day 5, compared to control group (ER^{T2} Et). Adjusted p values: ER^{T2} Et vs. ER^{T2} Tam *p=0.0251, ER^{T2} Et vs. Cre-ER^{T2} Et n.s. p>0.9999, ER^{T2} Et vs. Cre-ER^{T2} Tam ****p<0.0001. (6d) Intracellular staining for FoxO1 protein (left) and an extracellular staining of IL7R (right) after 72 hours of Cre-induction. The results are representative of 3 independent experiments. (6e) Luciferase bioimaging and (6f) Survival curves of mice that were injected with 1×10⁶ FoxO1^{fl/fl} cells that had been transformed with BCR-ABL1 and transduced with either a Cre-ER^{T2} construct or an empty vector control (ER^{T2}). Cells were labelled with luciferase and injected into NOD-SCID mice. (n=14 per group). Mantel-Cox-log-rank test, ***p=0.0002.

FIG. 7A to 7F: Anti-IL7R antibody suppresses BCR-ABL1+ALL development in vivo. (7a-7b) 1×10⁶ BCR-ABL1⁺ ALL patient cells were xenografted by tail vein injection into NSG mice. Xenografted mice were treated with vehicle, imatinib, or with anti-IL7R antibody (n=5 per group) as described in Methods. (7a) Survival prolongation in xenografted mice subjected to the indicated treatment. Mantel-Cox-log-rank test, **p=0.0027. (7b) Leukemic engraftment was measured by flow cytometry in peripheral blood (PB) at day 58 all control and imatinib-treated mice were sacrificed due to appearance of leukemic symptoms. Unpaired t-test, two-sided p value. (7c) Expression levels of ABL1 or BCR-ABL1 fusion in BCR-ABL1+ human cell lines (TOM-1 and SUP-15), and in imatinib-resistant xenograft-derived human BCR-ABL1+ cells. 697 cell line, was used as a negative control for the fusion. Expression levels are normalized to TOM-1. Error bars represent mean±SD. Unpaired t-test, two-sided. (7d-7f) 1×10⁶BCR-ABL1⁺ ALL patient cells holding T315I mutation (imatinib-resistant) were injected intravenously into NSG mice and treated with anti-IL7R or imatinib. (7d) Survival prolongation in mice xenografted with Ph+ BCP-ALL patient material holding the T315I mutation and treated with either imatinib or with anti-IL7R antibody. Mantel-Cox-log-rank test, **p=0.0027. (7e) Leukemic engraftment was measured by flow cytometry in PB at day 42. Unpaired t-test, two-sided. (7f) Spleen size at day 45 of xenograft mice treated with either imatinib or a combination of imatinib and IL7R antibody.

FIGS. 8A and 8B. Upregulation of CXCR4 signaling pathway in BCR-ABL1-transformed pre-B cells. (8a) Gene expression variances between BCR-ABL1 transformed cell lines or control cell lines expressing empty vector (EV) are displayed as three-dimensional principal component analysis (PCA) of RNA-seq data. Each sample is represented as a dot and localized on basis of its gene expression pattern. PCA plot was used as an additional quality control for the RNA-seq data. Of note, EV and BCR-ABL samples show dear segregation on the first PC that explained 57.12% of the variation in the dataset. (8b) Statistically significantly upregulated genesets related to IL7R signaling with False Discovery Rate (FDR)<0.25; other than JAK-STAT pathway shown in FIG. 1b. See Supplementary Table 2 for statistical details.

FIG. 9 Upregulation of CXCR4 signaling pathway in BCR-ABL1-transformed pre-B cells. Gene Set Enrichment Analysis (GSEA) showing upregulation of gene set belonging to the CXCR4 signaling pathway in BCR-ABL1 group. Heatmap representation (left) of the top 12 deregulated genes (core enrichment genes) in BCR-ABL1 versus EV-transduced samples (Blue: down-regulated; Red: up-regulated; NES=Normalized Enrichment Score; FDR=False Discovery Rate). A two-sided signal-to-noise metric was used to rank the genes. For a calculated GSEA nominal p-values of 0, we present them as p<0.001 (otherwise, exact p-values are shown). Multiple hypothesis testing correction is represented by the estimated FDR.

FIGS. 10A and 10B. Reduced IL7R and CXCR4 expression in BCR-ABL+ ALL as compared to other BCP-ALL subgroups. (10a) The expression levels of IL7R and CXCR4 in BCR-ABL+ ALL (t9; 22) (red) in comparison to other BCP-ALL entities (blue). The data are obtained from a mixed leukemia gene expression studyl; R2 database. Box and whisker plots (25-75 percentile) are generated in R2 platform (https://hgservertamc.nl/cgi-bin/r2/main.cgi) (10b) The expression levels of IL7R and CXCR4 in BCR-ABL positive ALL in comparison to other BCR-ABL negative BCP-ALL entities obtained from RNA-sequencing data of 1,223 patients.2 Exact p value for IL7R ****p=0.000000000002. (10a-10b) Values are depicted as box-whisker plots, the center line representing the median, box limits representing the 25th-75th percentile, whiskers depicting the 1.5× interquartile range and points representing outliers; unpaired, two-sided t-test.

FIGS. 11A to 11D: IL7 counteracts imatinib-induced cell death of BCR-ABL1+ cells. (11a) WT pre-B cells were transformed with BCR-ABL1 and were then treated with 5 μM imatinib for 15 hours. The expression levels of Jak1, and Stat5a were determined using qRT-PCR. N=6 per group, and error bars represent mean±SD. Unpaired t-test, two-sided. (11b) WT pre-B cells were transformed with BCR-ABL1 and were then treated with 1 μM imatinib for 15 hours in the presence of IL7, or CXCL12, or both IL7 and CXCL12, or with AMD3100 as indicated, then cell cycle analysis was performed. Quantification of percentages of cells in S-phase or GO/G1 is shown. AU: arbitrary unit. (n=4 per group), error bars represent mean±SD. Unpaired t-test, two-sided. GO/G1 (*p1=0.0286, *p2=0.0286, *p3=0.0286, *p4=0.0286, n.s. p5=0.8750); S phase (*p1=0.0286, *p2=0.0286, *p3=0.0286, p4=0.0286, n.s. p5=0.8750). (11c) BCR-ABL1-transformed WT pre-B cells were treated with 1 μM imatinib and with CXCL12 (left) or TSLP (right) at different concentrations as indicated for 6 days. The ratio of living cells was determined by flow cytometry. AU: arbitrary unit. (n=2 per group). (11d) Human Ph+ ALL SUP-B 15 cell line was treated with 5 μM imatinib and surface expression of IL7R (top) and CXCR4 (bottom) was measured by flow cytometry at day 7. The results are representative of 3 independent experiments.

FIGS. 12A to 12B: IL7R is indispensable for BCR-ABL1-derived transformation. (12a) Bone marrow-derived pre-B cells from either WT or IL7R@ knock out (KO) mice were transduced with either an empty vector (EV) or with BCR-ABL1. Viability of the cells after IL7 withdrawal was determined by flow cytometry (top) using Syotx as excluding dead cell stain. The percentages of lymphoid (CD19+GFP+) or myeloid (CD11b+GFP+) cells were analyzed (bottom). (12b) The enrichment of CD19+ cells in relative to control after IL7 withdrawal (n=2 per group). AU: arbitrary unit.

FIGS. 13A to 13D: CXCR4 expression is required for the survival of BCR-ABL1 transformed cells. (13a) CXCR4fl/fl pre-B cells were transformed with BCR-ABL1 and were then transduced with Cre-ERT2. Cells were treated with either tamoxifen (Tam) to induce Cre expression, or with ethanol (Et). PCR analysis for CXCR4 deletion (CXCR4Δ), SRp20 was used as a loading control. The results are representative of 3 independent experiments; bp: base pair. (13b) The surface expression of IL7R or CXCR4 after 24 hours of Tam induction. (13c) Colony formation assay for CXCR4^{fl/fl} cells which were transduced with BCR-ABL1 and Cre-ERT2. Cells were treated with either Et or Tam and incubated to allow colony formation for 3 weeks. N=4 independent samples per group, and error bars represent mean±SD. Unpaired t-test, two-sided. (13d) The percentage of living cells were determined by flow cytometry using Sytox as an excluding dead cell stain, after 72 hours of tamoxifen induction. N=6 independent samples per group, and error bars represent mean±SD. Unpaired t-test, two-sided.

FIGS. 14A to 14C: CXCR4 role in calcium mobilization and migration of BCR-ABL1 transformed cells. (14a) CXCR4^{fl/fl}-BCR-ABL1 cells containing Cre-ERT2 were induced with either ethanol (Et) or tamoxifen (Tam) for 48 hours, then CXCL12-induced calcium flux was measured. CXCR4 inhibition using AMD310 was used as a control. The results are representative of 3 independent experiments. (14b) Enrichment of differentiated cells (μ+kappa+) in CXCR4 deficient cells (n=2). (14c) CXCR4 or IL7Ra deletion was induced by tamoxifen for 24 hours and cells were then subjected to migration gradient toward CXCL12 (100 ng/ml) for 16 hours. The cells in the lower chamber were counted by trypan blue. N=6 per group, error bars represent mean±SD. Unpaired t-test, two-sided.

FIGS. 15A to 15F: CXCR4 and IL7R exist in dose proximity in both mouse and human BCR-ABL1+ALL cells. (15a) Flow cytometry staining of bone marrow-derived pre-B cells or mature splenic B cells which were used for PLA experiments in FIG. 4b. The results are representative of 3 independent experiments. (15b) PLA showing the loss of dose proximity between IL7R and CXCR4 in BCR-ABL1 transformed cells upon inducible deletion of CXCR4 using Cre-ERT2 system. Quantification of red dots (right). (15c) Fab-PLA analysis of IL7R-CXCR4 proximity in in human BCR-ABL+ cell line (SUP-B15) in comparison to Ramos cells. Quantification of red dots (middle). IL7R surface expression as measured by flow cytometry (left). (15d) SUP-B15 Ph+ ALL cells were lysed and an Immunoprecipitation (IP) was performed using an antibody against the BCR. IP with an isotype antibody was used as a negative control. The results are representative of 2 independent experiments; KDa: Kilo Dalton (15e) PLA showing that dose association between p-Jak3 and CXCR4 is reduced in BCR-ABL1 transformed cells upon inducible deletion of IL7R using Cre-ERT2 system. (15f) PLA showing that dose association between Jak3 and IL7R is not significantly different between WT pre-B cells and BCR-ABL1 transformed cells. (15b-15c, 15e-15f) Quantification shows number of signals per cell, error bars represent mean±SD. Unpaired t-test, two-sided. The results are representative of 3 independent experiments.

FIG. 16: Simplified graph for the proposed regulatory model. IL7R and CXCR4 synergize to facilitate BCR-ABL1-mediated pre-B cell transformation. In this platform, CXCR4 recruits BCR-ABL1 into dose proximity to the IL7R downstream signaling pathway (JAK/STAT) which results in STAT5 activation. STAT5 activation inhibits FOXO1 transcription factor, which in turn regulates IL7R expression.

FIG. 17A to 17F: Effects of ruxolitinib treatment in BCR-ABL+ ALL. (17a) WT pre-B cells transformed with BCR-ABL1 were treated with 1 μM imatinib and 2.5ng/ml IL7 in the presence of different concentrations of ruxolitinib and the fold change of living cells relative to control was determined by flow cytometry. N=3 independent samples per group, and error bars represent mean±SD. One-way ANAOVA. Dunnett's multiple comparisons test was performed to day 5, compared to control group (Vehicle). Adjusted p values: Veichle vs. 0.5 μM Ruxolitinib ****p<0.0001, Veichle vs. 1μM Ruxolitinib ****p<0.0001, Veichle vs. 5 μM Ruxolitinib****p<0.0001. Human BCR-ABL1+ cell lines TOM-1 (17b) and SUP-B15 (17c) were treated with vehicle only, imatinib only (2 μM), ruxolitinib only (10 μM) or combination of ruxolitinib and imatinib and the percentage of living cells were determined using Sytox viability dye (n=3 per group) relative to control. (17b-17c) N=3 independent samples per group, and error bars represent mean±SD. One-way ANAOVA. Dunnett's multiple comparisons test was performed to day 5, compared to control group (Vehicle). Adjusted p values (17b): Vehicle vs. Imatinib **p =0,0014, Vehicle vs. Ruxo****p<0.0001, Vehicle vs. Imatinib/Ruxo****p<0.0001. Adjusted p values (17c): Vehicle vs. Imatinib ***p<0.0001, Vehicle vs. Ruxo****p<0.0001, Vehicle vs. Imatinib/Ruxo****p<0.0001. (17d) SUP-B15 ALL cells were injected into NSG mice and the mice were subjected to treatment with vehicle only, imatinib only, ruxolitinib only or combination of ruxolitinib and imatinib (n=5 per group) as described in Methods. Survival prolongation in xenografted mice subjected to the indicated treatment. Statistics for survival were performed according to the Mantel-Cox log-rank method and showed no significant statistical difference among the groups (e) Spleen sizes from the corresponding groups. (17f) Leukemic engraftment was measured by flow cytometry in BM and spleen. Unpaired t-test, two-sided, no significant statistical difference among the groups when compared to control group (vehicle).

FIGS. 18A to 18C: in vivo engraftment of imatinib-resistant BCR-ABL+ ALL. Xenograft mice of an imatinib-resistant BCR-ABL patient material were generated and treated with either vehicle or imatinib (n=5 per group) as described in FIG. 7a. (18a) A representative flow cytometry staining of leukemic blasts derived from bone marrow (BM) or spleen (Sp) or (18b) peripheral blood at day 58. The percentage of leukemic cells (positive for human CD19) are indicated in the lower right quadrant of each plot. (18c) Spleen size of xenografted mice treated with imatinib or with imatinib and IL7R antibody as described in FIG. 7f. Unpaired t-test, two-sided.

FIGS. 19A to 19C: Upregulation of BCR-ABL can lead to imatinib-resistance in BCR-ABL+ transformed pre-B cells. Mouse WT pre-B cells transformed with BCR-ABL1 were treated with either vehicle or imatinib (1 μIV) for 18 days to induce imatinib-resistance. (19a) A schematic diagram showing the time schedule for adding or washing-off imatinib. (19b) Expression levels of BCR-ABL1 fusion in five different WT-BCR-ABL transformed pre-B cells as measured by quantitative RT-PCR. Expression levels are normalized to cells treated with vehicle (DMSO). (19c) A summary graph for the upregulation of BCR-ABL of all cell lines. Paired t-test, two-sided p value.

FIGS. 20A to 20C: Anti-IL7R antibody induces apoptosis. (20a) A representative extracellular flow cytometry staining showing that anti-IL7R antibody used for in vivo treatment (in FIG. 7) does not bind to the ligand binding site of IL7R. Sup-B15 cells were incubated with human IL7 for 15 minutes, then were treated with two different anti-IL7R antibodies (left: from R&D; right: from BioLegend) for 15 minutes on ice. A secondary antibody was used when required. (20b) Fab-PLA analysis of extracellular IL7R-CXCR4 proximity in SUP-B15 which were treated with 10 μg/ml anti-IL7R antibody for 45 minutes at 37° C. in comparison to untreated cells. Quantification of red dots shows number of signals per cell (top), error bars represent mean±SD. Unpaired t-test, two-sided, p<0.000000000000001. IL7R and CXCR4 surface expression as measured by flow cytometry after the treatment (bottom). The results are representative of 3 independent experiments. (20c) Anti-IL7R antibody treatment leads to apoptosis. Two different BCR-ABL1+ patient derived xenograft (PDX) ALL cells were treated 10 μg of anti-IL7R antibody for 45 minutes. Cell lysates were subjected to western blotting and Caspase-8 levels were detected. Treatment with anti-IL7R antibody activates caspase-8 cleavage and leads to the release of the caspase-8 active fragment p18. The results are representative of 2 independent experiments. KDa: Kilo Dalton.

FIG. 21: Gating strategies used for flow cytometry analysis. (21a) Lymphocytes gate was analyzed depending on distinguished FSC vs. SSC properties. Singlets were then selected (FSC-W vs FSC-H then SSC-W vs SSC-H). The cells were then further analyzed according to their surface or intracellular protein stains. Similar gating strategy was applied to Figures. 2b, 3a, 3d, 5c, 5e, 6d, 7b, 7e, 11b, 11d, 13b, 14b, 15a, 15c, 17f, 18a-b,and 20a-b. (21b) For viability experiments, singlets were first selected (FSC-A vs FSC-H). Viable cells were then determined according to FSC-A and viability dye properties. Similar gating strategy was applied to Figures: 2d-e, 3b, 3e, 6b-c, 11c, 12, 13d, and 17a-c. (21c) For calcium analysis, lymphocytes gate was analyzed depending on distinguished FSC vs. SSC properties. Singlets were then selected (FSC-W vs FSC-H then SSC-W vs SSC-H). The cells stained with Indo-1 were then selected and then calcium kinetics were then shown relative to time. Similar gating strategy was applied to Figures: 4a and 14a.

FIGS. 22A to 22G: Uncropped original gel scans. Original Gel blots for FIG. 4a (a), FIG. 5a (b), FIG. 5b (c), FIG. 15(d) and FIG. 20(c). In some cases membranes were cut at certain sizes to allow blotting with different antibodies to avoid background resulting from multiple developments on the same membrane.

FIG. 23 shows the sequences of variable heavy chains and variable light chains. The CDRs (using IMGT numbering) are identified with underlining.

FIGS. 24A to 24C show that the anti-IL7R antibodies of the invention induce apoptosis. (25a) A representative extracellular flow cytometry staining showing that anti-IL7R antibody used for in vivo treatment (in FIG. 7) does not bind to the ligand binding site of IL7R. Ph+Sup-B15 BCP-ALL cells were incubated with human IL7 for 15 minutes, then were treated with two different anti-IL7R antibodies (left: from R&D; right: from BioLegend) for 15 minutes on ice. A secondary antibody was used when required. (b) Fab-PLA analysis of extracellular IL7R-CXCR4 proximity in SUP-B15 which were treated with 10 μg/ml anti-IL7R antibody for 45 minutes at 37° C. in comparison to untreated cells. Quantification of red dots shows number of signals per cell, error bars represent mean±SD. Unpaired t-test, two-sided, p<0.0500. (top). IL7R and CXCR4 surface expression as measured by flow cytometry after the treatment (bottom). (c) Anti-IL7R antibody treatment leads to apoptosis. Two different BCR-ABL1+ patient derived xenograft (PDX) ALL cells were treated 10 μg of anti-IL7R antibody for 45 minutes. Cell lysates were subjected to western blotting and Caspase-8 levels were detected. Treatment with anti-IL7R antibody activates caspase-8 cleavage and leads to the release of the caspase-8 active fragment p18. The results are representative of 2 independent experiments. KDa: Kilo Dalton.

FIG. 25 shows that the anti-IL7R antibodies of the invention lead to apoptosis but do not change Stat5 phosphorylation. This is interesting as it suggests that the antibody leads to cell death directly by activating downstream apoptotic pathways independent of the Stat5 pathway, taking into consideration that the latter is a common survival pathway.

FIG. 26: both WT-pre B cells and BCR-ABL1 transformed cells were cultured for similar times. BM cells were isolated from 3 different mice and then were kept in culture with IL7 for 7 days. Afterwards, pre-B cells were transduced with either EV or with BCR-ABL and kept for 48 hours in +IL7 medium. Then, IL7 was removed from cells transduced with BCR-ABL1 and cells were cultured in absence of IL7 for 1 week until cells were completely transformed as shown in the figure. To confirm transformation, cells transduced with EV were used as a control, as shown in the figure these cells die in the absence of IL7.

FIG. 27: CXCR4 is absolutely required for cell survival. As shown in the figure, deletion of CXCR4 in transformed BCR-ABL1 cells led to a rapid cell death. This effect could be reversed only by ectopic expression of survival factors, such as overexpressing BCL2.

FIG. 28: Statistically significantly upregulated genesets related to IL7R. FDR<0.25; highlighted in green. A two-sided signal-to-noise metric was used to rank the genes. For a calculated GSEA nominal p-values of 0, we present them as p<0.001 (otherwise, exact p-values are shown). Multiple hypothesis testing correction is represented by the estimated FDR.

FIG. 29: Clinical parameters of 68 BCR-ABL positive patients at initial diagnosis. 1SR - standard risk, IR -intermediate risk, HR - high risk. Risk stratification according to minimal residual disease (MRD) risk groups: MRD-SR: TP1+2 negative, MRD-IR: TP1 and/or TP2<10-3, MRD-HR: TP2 10-3. MRD risk group was missing for 2 patients in the CNS pos. Prednisone poor responders were stratified into the HR treatment group. * Mann-Whitney test, 2-sided P value. ‡1-way ANOVA.

FIGS. 30A to 30B: additional information to Boxplots in FIGS. 10A and 10B.

FIGS. 31A to 31C: list of all antibodies used in the disclosure herein.

FIGS. 32: exemplary IgG formats and associated sequences.

### DETAILED DESCRIPTION OF THE INVENTION

A. Definitions

In order that the application may be more completely understood, several definitions are set forth below. Such definitions are meant to encompass grammatical equivalents.

By “amino acid” and “amino acid identity” as used herein is meant one of the 20 naturally occurring amino acids or any non-natural analogues that may be present at a specific, defined position. In many embodiments, “amino acid” means one of the 20 naturally occurring amino acids. By “protein” herein is meant at least two covalently attached amino acids, which includes proteins, polypeptides, oligopeptides and peptides.

By “amino acid modification” herein is meant an amino acid substitution, insertion, and/or deletion in a polypeptide sequence or an alteration to a moiety chemically linked to a protein. For example, a modification may be an altered carbohydrate or PEG structure attached to a protein. For clarity, unless otherwise noted, the amino acid modification is always to an amino acid coded for by DNA, e.g. the 20 amino acids that have codons in DNA and RNA. The preferred amino acid modification herein is a substitution.

By “amino acid substitution” or “substitution” herein is meant the replacement of an amino acid at a particular position in a parent polypeptide sequence with a different amino acid. In particular, in some embodiments, the substitution is to an amino acid that is not naturally occurring at the particular position, either not naturally occurring within the organism or in any organism. For clarity, a protein which has been engineered to change the nucleic acid coding sequence but not change the starting amino acid (for example exchanging CGG (encoding arginine) to CGA (still encoding arginine) to increase host organism expression levels) is not an “amino acid substitution”; that is, despite the creation of a new gene encoding the same protein, if the protein has the same amino acid at the particular position that it started with, it is not an amino acid substitution.

By “amino acid insertion” or “insertion” as used herein is meant the addition of an amino acid sequence at a particular position in a parent polypeptide sequence.

By “amino acid deletion” or “deletion” as used herein is meant the removal of an amino acid sequence at a particular position in a parent polypeptide sequence.

The polypeptides of the invention specifically bind to human IL7R as outlined herein. “Specific binding” or “specifically binds to” or is “specific for” a particular antigen or an epitope means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

Specific binding for a particular antigen or an epitope can be exhibited, for example, by an antibody having a KD for an antigen or epitope of at least about 10⁻⁴M, at least about 10⁻⁵ M, at least about 10⁻⁶ M, at least about 10⁻⁷M, at least about 10⁻⁸M, at least about 10⁻⁹M, alternatively at least about 10⁻¹⁰ M, at least about 10⁻¹¹ M, at least about 10⁻¹² M, or greater, where KD refers to a dissociation rate of a particular antibody-antigen interaction. Typically, an antibody that specifically binds an antigen will have a KD that is 20-, 50-, 100-, 500-, 1000-, 5,000-, 10,000- or more times greater for a control molecule relative to the antigen or epitope.

Also, specific binding for a particular antigen or an epitope can be exhibited, for example, by an antibody having a KA or Ka for an antigen or epitope of at least 20-, 50-, 100-, 500-, 1000-, 5,000-, 10,000- or more times greater for the epitope relative to a control, where KA or Ka refers to an association rate of a particular antibody-antigen interaction. Binding affinity is generally measured using a Biacore assay or Octet as is known in the art.

By “position” as used herein is meant a location in the sequence of a protein. Positions may be numbered sequentially, or according to an established format, for example the EU index for antibody numbering.

By “Fv” or “Fv domain” or “Fv region” as used herein is meant a polypeptide that comprises the VL and VH domains of an antigen binding domain, generally from an antibody. Fv domains usually form an “antigen binding domain” or “ABD” as discussed herein.

By “variable domain” herein is meant the region of an immunoglobulin that comprises one or more Ig domains substantially encoded by any of the Vx, VA, and/or VH genes that make up the kappa, lambda, and heavy chain immunoglobulin genetic loci respectively. In some cases, a single variable domain, such as an sdFv (also referred to herein as sdABD) can be used.

In embodiments utilizing both variable heavy (VH) and variable light (VL) domains, each VH and VL is composed of three hypervariable regions (“complementary determining regions,” “CDRs”) and four “framework regions”, or “FRs”, arranged from amino-terminus to carboxy-terminus in the following order: FR1-CDR1-FR2-CDR2-FR3-CDR3-FR4. Thus, the VH domain has the structure vhFR1-vhCDR1-vhFR2-vhCDR2-vhFR3-vhCDR3-vhFR4 and the VL domain has the structure vlFR1-vlCDR1-vlFR2-vlCDR2-vlFR3-vlCDR3-vlFR4. As is more fully described herein, the vhFR regions and the vlFR regions self assemble to form Fv domains.

The hypervariable regions confer antigen binding specificity and generally encompasses amino acid residues from about amino acid residues 24-34 (LCDR1; “L” denotes light chain), 50-56 (LCDR2) and 89-97 (LCDR3) in the light chain variable region and around about 31-35B (HCDR1; “H” denotes heavy chain), 50-65 (HCDR2), and 95-102 (HCDR3) in the heavy chain variable region; Kabat et al., SEQUENCES OF PROTEINS OF IMMUNOLOGICAL INTEREST, 5th Ed. Public Health Service, National Institutes of Health, Bethesda, Md. (1991) and/or those residues forming a hypervariable loop (e.g. residues 26-32 (LCDR1), 50-52 (LCDR2) and 91-96 (LCDR3) in the light chain variable region and 26-32 (HCDR1), 53-55 (HCDR2) and 96-101 (HCDR3) in the heavy chain variable region; Chothia and Lesk (1987) J. Mol. Biol. 196:901-917. Specific CDRs of the invention are described below, and utilize the IMGT numbering system for CDR placement.

As will be appreciated by those in the art, the exact numbering and placement of the CDRs can be different among different numbering systems. However, it should be understood that the disclosure of a variable heavy and/or variable light sequence includes the disclosure of the associated (inherent) CDRs. Accordingly, the disclosure of each variable heavy region is a disclosure of the vhCDRs (e.g. vhCDR1, vhCDR2 and vhCDR3) and the disclosure of each variable light region is a disclosure of the vlCDRs (e.g. vlCDR1, vlCDR2 and vlCDR3).

A useful comparison of CDR numbering is as below, see Lafranc et al., Dev. Comp. Immunol. 27(1):55-77 (2003):

TABLE 1

| | Kabat + | | | | | |
|---|---|---|---|---|---|---|
| | Chothia | IMGT | Kabat | AbM | Chothia | Contact |
| vhCDR1 | 26-35 | 27-38 | 31-35 | 26-35 | 26-32 | 30-35 |
| vhCDR2 | 50-65 | 56-65 | 50-65 | 50-58 | 52-56 | 47-58 |
| vhCDR3 | 95-102 | 105-117 | 95-102 | 95-102 | 95-102 | 93-101 |
| vlCDR1 | 24-34 | 27-38 | 24-34 | 24-34 | 24-34 | 30-36 |
| vlCDR2 | 50-56 | 56-65 | 50-56 | 50-56 | 50-56 | 46-55 |
| vlCDR3 | 89-97 | 105-117 | 89-97 | 89-97 | 89-97 | 89-96 |

The present invention provides a number of different CDR sets. In this case, a “full CDR set” in the context of the anti-CD3 component comprises the three variable light and three variable heavy CDRs, e.g. a vlCDR1, vlCDR2, vlCDR3, vhCDR1, vhCDR2 and vhCDR3. As will be appreciated by those in the art, each set of CDRs, the VH and VL CDRs, can bind to antigens, both individually and as a set

These CDRs can be part of a larger variable light or variable heavy domain, respectfully. In addition, as more fully outlined herein, the variable heavy and variable light domains can be on separate polypeptide chains or on a single polypeptide chain in the case of scFv sequences, depending on the format and configuration of the moieties herein.

The CDRs contribute to the formation of the antigen-binding, or more specifically, epitope binding sites. “Epitope” refers to a determinant that interacts with a specific antigen binding site in the variable regions known as a paratope. Epitopes are groupings of molecules such as amino acids or sugar side chains and usually have specific structural characteristics, as well as specific charge characteristics. A single antigen may have more than one epitope.

The epitope may comprise amino acid residues directly involved in the binding (also called immunodominant component of the epitope) and other amino acid residues, which are not directly involved in the binding, such as amino acid residues which are effectively blocked by the specific antigen binding peptide; in other words, the amino acid residue is within the footprint of the specific antigen binding peptide.

Epitopes may be either conformational or linear. A conformational epitope is produced by spatially juxtaposed amino acids from different segments of the linear polypeptide chain. A linear epitope is one produced by adjacent amino acid residues in a polypeptide chain. Conformational and nonconformational epitopes may be distinguished in that the binding to the former but not the latter is lost in the presence of denaturing solvents.

An epitope typically includes at least 3, and more usually, at least 5 or 8-10 amino acids in a unique spatial conformation. Antibodies that recognize the same epitope can be verified in a simple immunoassay showing the ability of one antibody to block the binding of another antibody to a target antigen, for example “binning.” As outlined below, the invention not only includes the enumerated antigen binding domains and antibodies herein, but those that compete for binding with the epitopes bound by the enumerated antigen binding domains.

B. Introduction

The Philadelphia chromosome (Ph) is the most frequent abnormality among adults with acute lymphoblastic leukemia (ALL) (25%-30%) and results in the creation of a BCR-ABL1 fusion gene¹. Furthermore, 3-5% of children harbor this translocation, which is associated with a poor prognosis^{2,3}. As this oncogene confers constitutive kinase activity, addition of tyrosine kinase inhibitors (TKIs) such as imatinib mesylate to intensive chemotherapy has improved the outcome of BCR-ABL1-positive leukemia to a 5-year disease-free survival rate in children (70%±12%, n=28)³. Nevertheless, Ph+ ALL patients still suffer from poor prognosis in both children and adults as relapse frequently occurs even after stem cell transplantation. A deep understanding of the molecular mechanisms which are associated with BCR-ABL1 transformation is of high importance in order to provide better treatment for these patients and to overcome TKI-resistance. Recently, our group has shown that interleukin 7 receptor (IL7R) is widely expressed in B cell precursor-ALL (BCP-ALL), and that high expression levels of IL7R are correlated with central nervous system involvement (CNS) and may predict CNS-relapse⁴.

The cytokine IL7 binds to IL7Rα chain that heterodimerizes with the common gamma chain (γc) to form the IL7 receptor and induces the kinase activity of JAK1/JAK3⁵. Alternatively, the IL7Rα chain hetero-dimerizes with the cytokine receptor-like factor 2 (CRLF2) to form the thymic stromal lymphopoietin receptor (TSLPR) and mediate activation of JAK1/JAK2⁶. The constitutive expression of IL7R^{7,8} in ALL together with the high frequency of mutations affecting IL7R signaling point to a key role of IL7R in disease pathogenesis⁹⁻¹¹. Thus, investigating the regulation of IL7R function is important for understanding its role in the pathogenesis of ALL. Moreover, characterizing the molecular interaction of IL7R might provide crucial insights into the mechanisms of malignant transformation.

Available data suggest that IL7R expression is controlled by the Forkhead box transcription factor 1 (FoxO1) in lymphocytes¹². Importantly, FoxO1 is essential during early B cell development and its activity is negatively regulated by phosphatidyinositol-3-kinase (PI3K) signaling¹³. Therefore, FoxO1 function depends on the lipid phosphatase PTEN (phosphatase and tensin homologue) which counteracts PI3K function¹⁴.

The C—X—C chemokine receptor 4 (CXCR4) is a G-protein-coupled receptor which is widely expressed on hematopoietic stem cells and hematopoietic cancers. Together with its ligand CXCL12 (also known as stromal-derived factor 1; SDF1), CXCR4 plays an important role in tumorigenesis by regulating survival, migration, homing and interaction of leukemia cells with their microenvironment¹⁵. High CXCR4 protein expression is correlated with an increased risk of relapse and poor outcome in pediatric ALL patients¹⁶. Interestingly, CXCL12 was initially identified as a soluble factor that collaborates with IL7 to activate the proliferation of progenitor B cells^{17,18}.

In this study we have investigated the molecular mechanisms, which are regulated by the oncogenic kinase BCR-ABL1 and are required for malignant transformation or for rescue from kinase inhibitor treatment. We show that IL7R and CXCR4 interact on the cell surface and that both are crucial for malignant transformation of early B cells by BCR-ABL1. Importantly, we show that anti-IL7R antibody can efficiently eliminate inhibitor-resistant Ph+ patient ALL in preclinical xenograft models.

Treatment with BCR-ABL1 kinase inhibitors result in elevated expression of IL7R which enables the survival of transformed cells when IL7 was added together with the kinase inhibitors. Importantly, we show treatment with anti-IL7R antibodies prevents leukemia development in xenotransplantation models using patient-derived Ph+ ALL cells.

Our results suggest that the association between IL7R and CXCR4 serves as molecular platform for BCR-ABL1 induced transformation and development of Ph+ ALL. Targeting this platform with anti-IL7R antibody eliminates Ph+ ALL cells including those with resistance to commonly used ABL1 kinase inhibitors. Thus, anti-IL7R antibodies may provide alternative treatment options for ALL in general and may suppress incurable drug-resistant leukemia forms.

C. Anti-IL7R Antibodies

Accordingly, the present invention provides antibodies that bind to human IL7R but do not prevent binding of human IL7R to human IL7.

In some embodiments, the antibodies provided herein do not bind to the ligand binding site of IL7R.

In some embodiments, the antibodies provided herein bind to the ligand binding site of IL7R.

1 Variable Domains

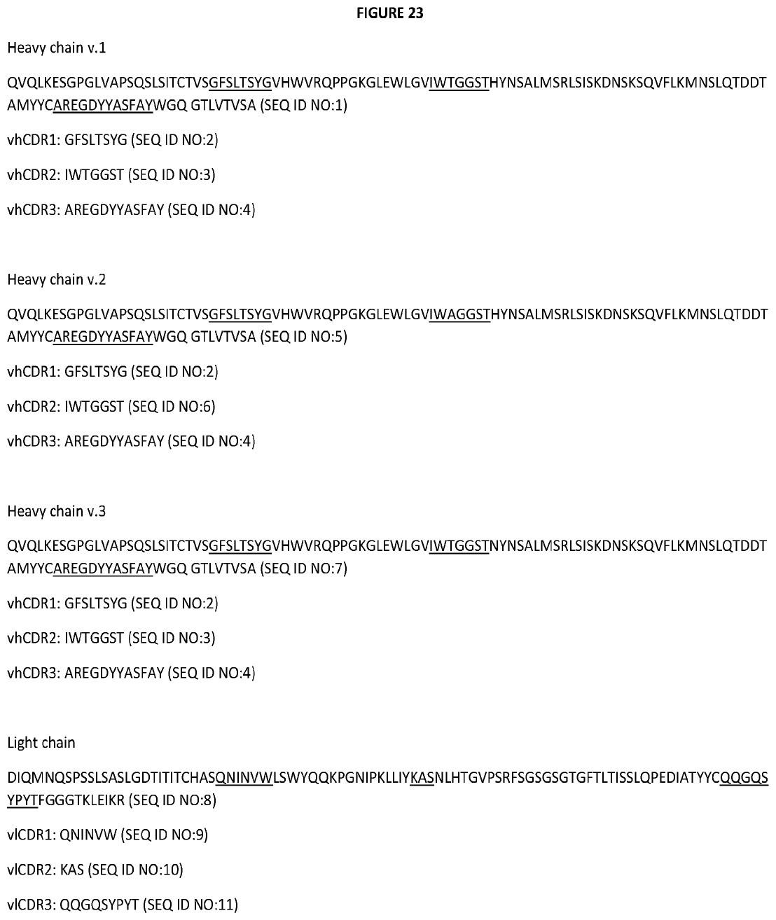

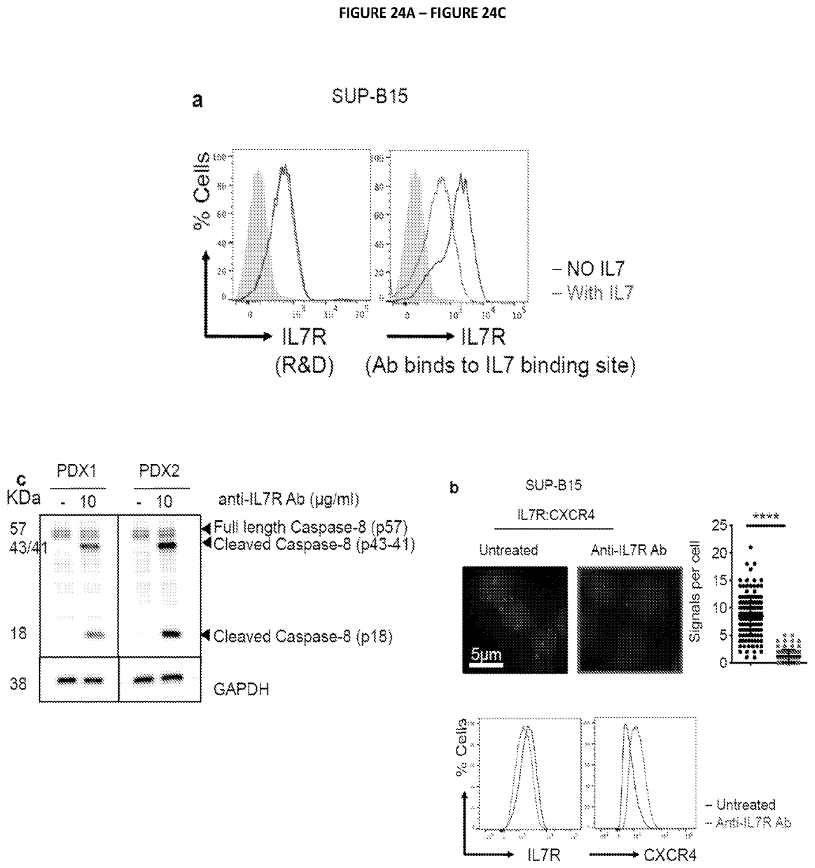

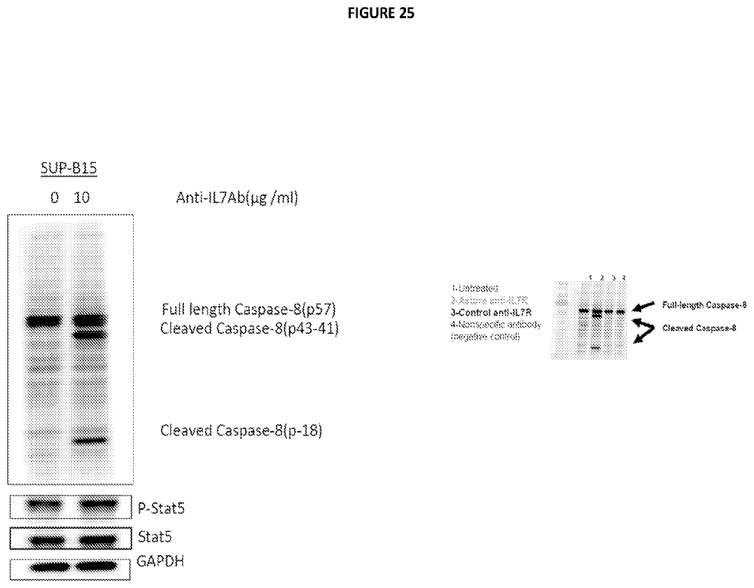

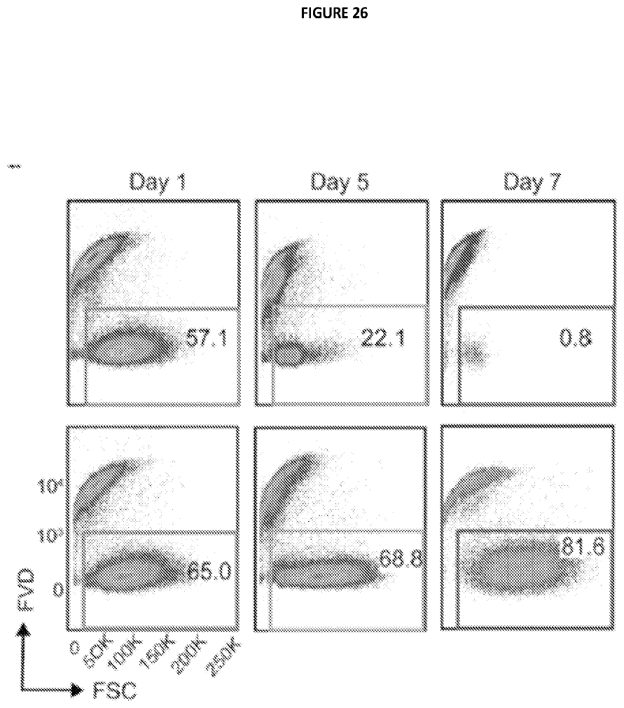

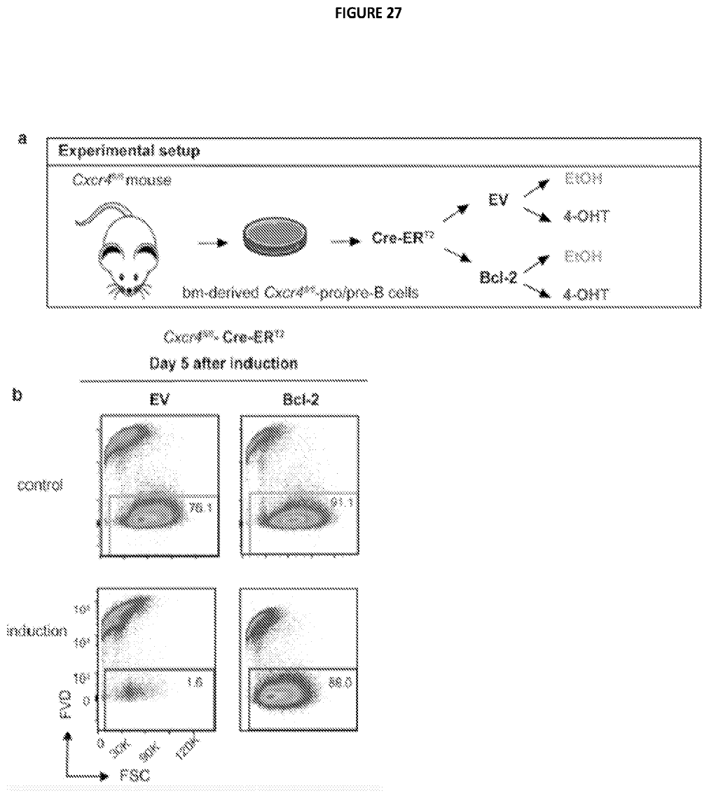

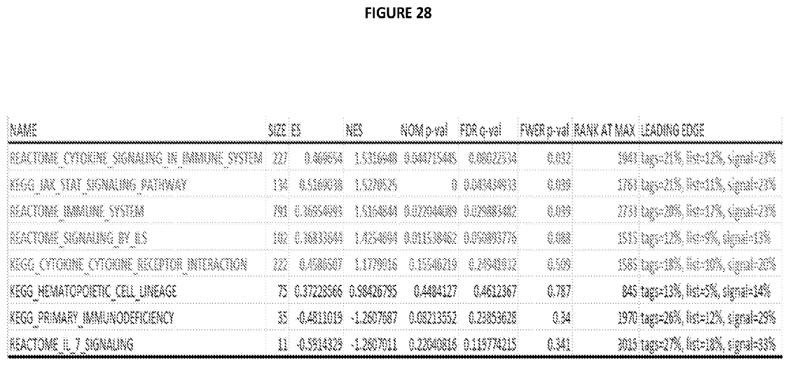

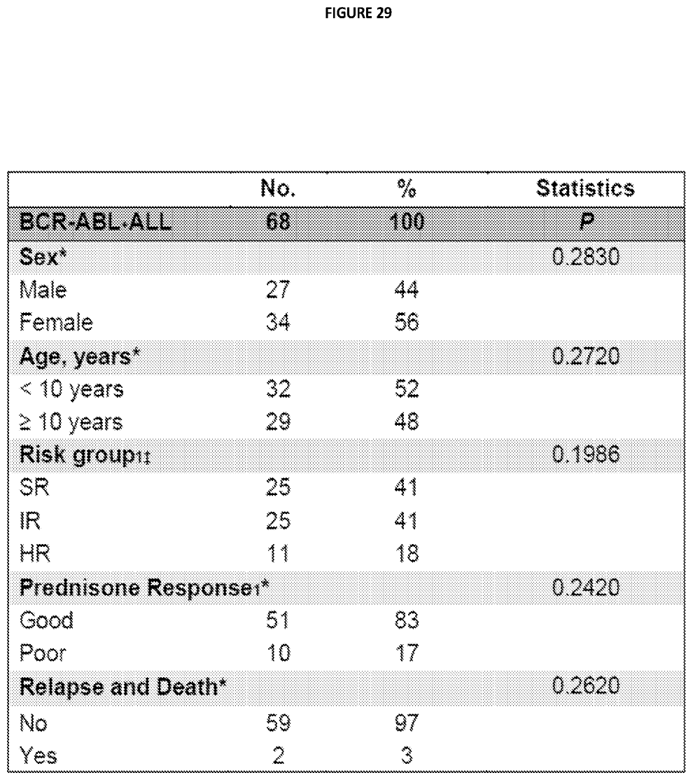

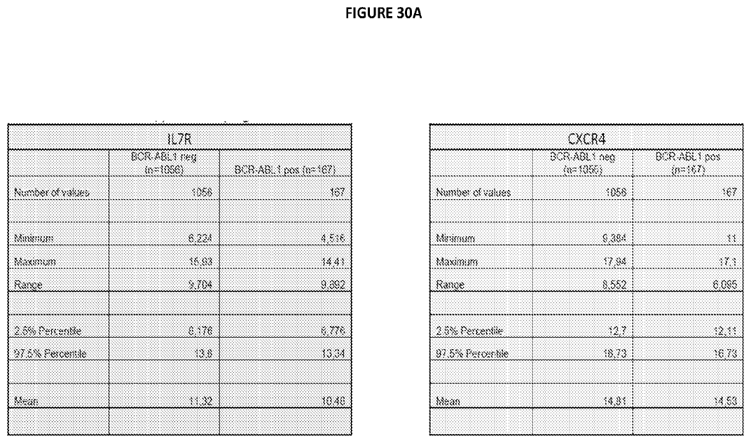

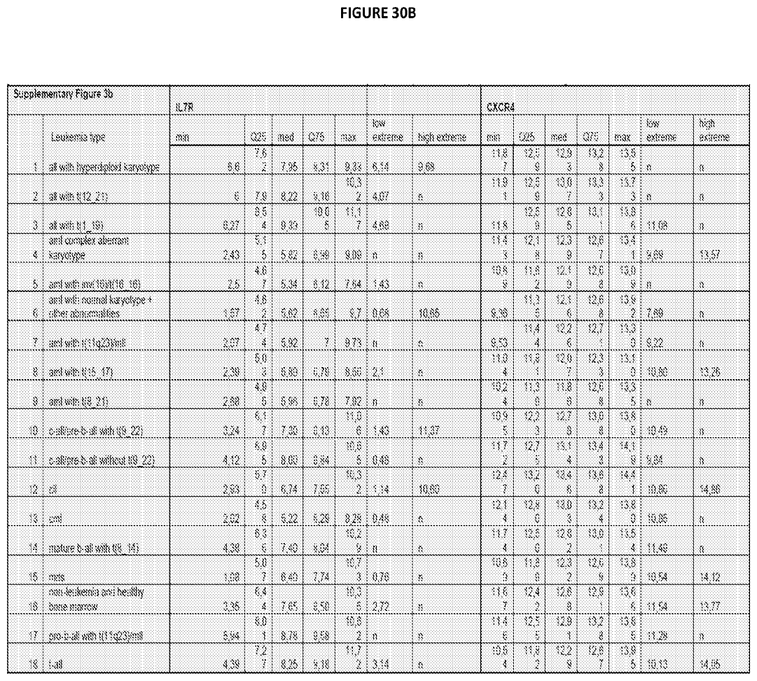

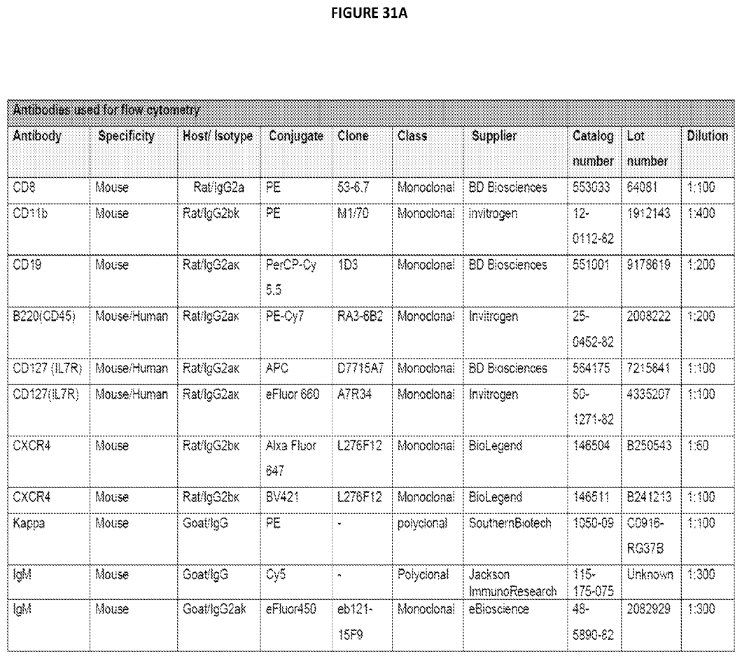

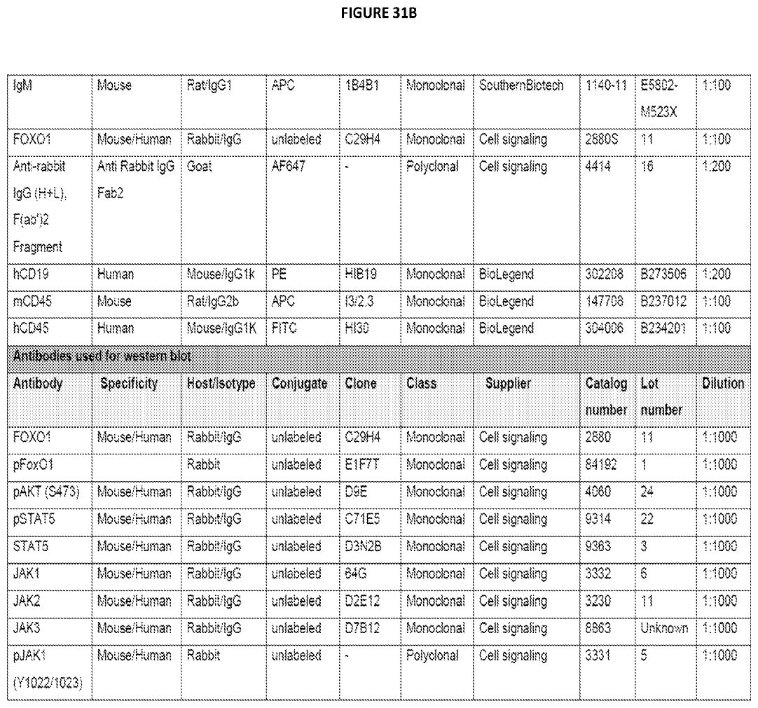

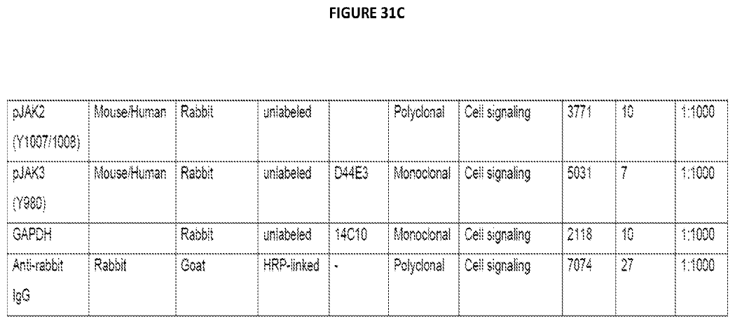

The present invention provides CDR sets as well as variable heavy and variable light domains as depicted in FIG. 23. Thus, antibodies are provided that contain vhCDRs of SEQ ID Nos:2, 3 and 4 in combination with v1CDRs having SEQ ID Nos:9, 10 and 11. Thus, antibodies are provided that contain vhCDRs of SEQ ID Nos:2, 6 and 4 in combination with v1CDRs having SEQ ID Nos:9, 10 and 11.

In some embodiments, the antibodies provided contain vhCDRs of SEQ ID Nos:2, 3 and 4, with optionally 1 to 3 amino acid substitutions per CDR, and exhibit 80%, 85%, 90%, 95%, or 99% identity to SEQ ID No:1. In some embodiments, the antibodies provided contain vhCDRs of SEQ ID Nos: 2, 6 and 4, with optionally 1 to 3 amino acid substitutions per CDR, and exhibit 80%, 85%, 90%, 95%, or 99% identity to SEQ ID No:5. In some embodiments, the antibodies provided contain vhCDRs of SEQ ID Nos: 2, 3 and 4, with optionally 1 to 3 amino acid substitutions per CDR, and exhibit 80%, 85%, 90%, 95%, or 99% identity to SEQ ID No:7. In some embodiments, the antibodies provided contain v1CDRs of SEQ ID Nos: 9, 10 and 11, with optionally 1 to 3 amino acid substitutions per CDR, and exhibit 80%, 85%, 90%, 95%, or 99% identity to SEQ ID No:8.

Similarly, antibodies are provided that contain vhCDRs of SEQ ID Nos:2, 6 and 4 in combination with v1CDRs having SEQ ID Nos:9, 10 and 11.

In some embodiments, the antibodies comprise a heavy chain exhibiting 80%, 85%, 90%, 95%, or 99% identity to SEQ ID No: 1, 5, or 7. In some embodiments, the antibodies comprise a heavy chain exhibiting 80%, 85%, 90%, 95%, or 99% identity to SEQ ID No:8.

2. Constant Domains

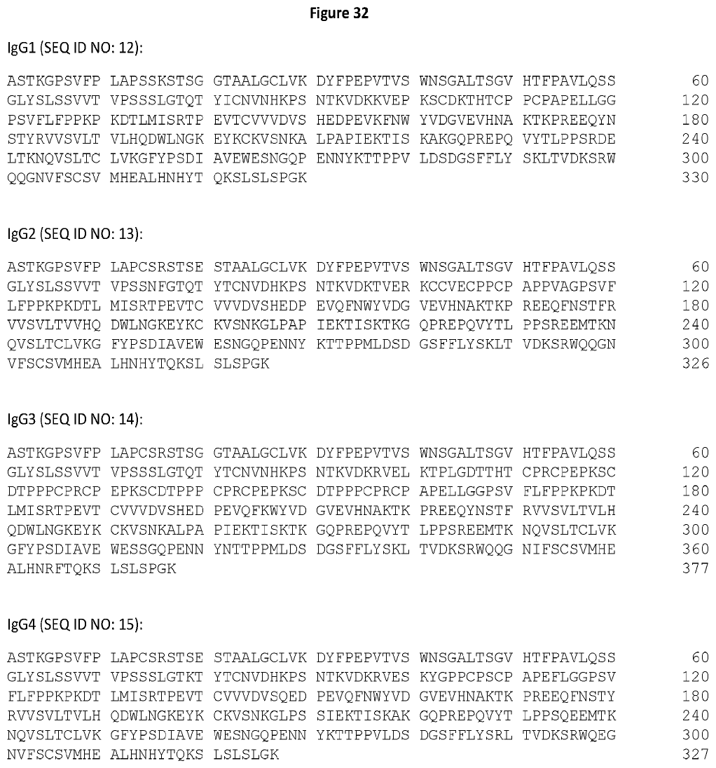

As will be appreciated by those in the art, CDR sets and/or variable heavy and variable light domains can be combined with human IgG1, IgG2 or IgG4 constant domains. Exemplary sequences can be found in SEQ ID No:12-15, as provided in FIG. 32.

D. Nucleic Acids, Expression Vectors and Host Cells

The invention further provides nucleic acid compositions encoding the anti-IL7R antibodies. In general, as known in the art, nucleic acids are provided that encode the heavy chain and the light chain. These nucleic acids can be incorporated into one expression vector or two.

As is known in the art, the nucleic acids encoding the components of the invention can be incorporated into expression vectors as is known in the art, and depending on the host cells used to produce the antibodies of the invention. Generally the nucleic acids are operably linked to any number of regulatory elements (promoters, origin of replication, selectable markers, ribosomal binding sites, inducers, etc.). The expression vectors can be extra-chromosomal or integrating vectors.

The nucleic acids and/or expression vectors of the invention are then transformed into any number of different types of host cells as is well known in the art, including mammalian, bacterial, yeast, insect and/or fungal cells, with mammalian cells (e.g. CHO cells), finding use in many embodiments.

In some embodiments, nucleic acids encoding the heavy and light chains are each contained within a single expression vector, generally under different or the same promoter controls. In embodiments of particular use in the present invention, each of these two nucleic acids are contained on a different expression vectors.

The anti-IL7R antibodies of the invention are made by culturing host cells comprising the expression vector(s) as is well known in the art. Once produced, traditional antibody purification steps are done.

E. Formulations

Formulations of the antibodies used in accordance with the present invention are prepared for storage by mixing an antibody having the desired degree of purity with optional pharmaceutically acceptable carriers, excipients or stabilizers (Remington's Pharmaceutical Sciences 16th edition, Osol, A. Ed. [1980]), in the form of lyophilized formulations or aqueous solutions.

F. Methods of Treating Ph+ALL

As will be appreciated by those in the art, the anti-IL7R antibodies find use in several methods.

In some embodiments, the anti-IL7R antibodies are used as a monotherapy to treat Ph+ALL patients.

In some embodiments, the anti-IL7R antibodies are used in combination with standard chemotherapy. In these embodiments, the anti-IL7R antibodies can be administered prior to, simultaneously with, or subsequent to the administration of the chemotherapeutic agent(s).

In some embodiments, the anti-IL7R antibodies are used in combination with a kinase inhibitor such as imatinib. In these embodiments, the anti-IL7R antibodies can be administered prior to, simultaneously with, or subsequent to the administration of the imatinib.

In some embodiments, the patient has circulating malignant cells. In some embodiments, the antibodies will be used to treat patients who have relapsed following TKI therapy and in whom the circulating malignant cells are expressing IL7R.

### EXAMPLES

### A. Example 1

### Methods

1. Patient Samples, Human Cell Lines, and Mouse Cells.

68 BCR-ABL+ ALL patients were treated according to European intergroup study of post-induction treatment of Philadelphia-chromosome-positive ALL (EsPhALL) 2004 and 2010 protocols (NCT00287105) and ALL-Berlin-Frankfurt-Münster (BFM) 2000 (NCT00430118) study. Informed consent was obtained according to institutional regulations, in accordance with the Declaration of Helsinki. 697, SUP-B15, and TOM-1 cell lines were obtained from DSMZ. Ph+ ALL cells containing T3151 mutation was kindly provided by M. Miischen (Shojaee, S., et al. Nat Med 22, 379-387 (2016)).

2. Mice

All mouse housing, breeding, and surgical procedures were approved by the governmental institutions of Baden-Württemberg (Regierungsprasidium Tübingen). BM cells from WT (n=7, female), IL7Rα^{Δ} (n=7, female), IL7Rα^{fl/fl} (n=7, female), CXCR4^{fl/fl} (n=3, female) and FoxO1^{fl/fl} (n=3, female) mice were collected and retrovirally transformed with either an empty pMIG vector or with a pMIG vector expressing BCR-ABL1. Unless mentioned otherwise, cells were cultured for 3 to 7 days in Iscove's medium (Biochrom AG) containing 10% heat-inactivated FCS (Sigma-Aldrich), 2 mM 1-glutamine, 100 U/ml penicillin (Gibco), 100 U/ml streptomycin (Gibco), and 50 μM 2-mercaptoethanol. The medium was supplemented in excess with the supernatant of J558L plasmacytoma cells stably transfected with a vector encoding murine IL7. Transformed cells were selected by IL7 withdrawal and kept in optimum conditions³⁸. Retroviral vectors containing either constitutively active STAT5 (STAT5-CA)³⁴ or an empty vector were used to transduce BCR-ABL1-transformed cells and sorted cells were used then for western blot or flow cytometry analysis. 1-2μM 4-hydroxy tamoxifen (Tam) (Sigma-Aldrich) was used to induce deletion on plasmids expressing tamoxifen-inducible form of Cre (Cre-ER^{T2})¹⁴. All cells were tested and found free from mycoplasma.

3. Expression Assays

Total RNA was isolated using Direct-zol™ RNA Kit (Zymo Research) or ReliaPrep™ RNA Cell Miniprep System (Promega), and synthesis of cDNA was performed (Thermofisher). Quantitative real time PCR (qRT-PCR) analyses were performed on ABI7900HT PCR machine (Applied Biosystems) using Quantitect assays (Qiagen) and SYBR Green (Applied Biosystems). The expression of ABL1 and the fusion BCR-ABL (m-bcr; e1-a2) were measured using TaqMan Gene expression assays (Hs01104728_ml ABL1 and Hs03024844_ft BCR-ABL, respectively) from Applied Biosystems. Relative quantification (RQ) was calculated using 2\hat{ }-ΔCCT equation.

4. RNA-Seq

BM cells were isolated from 3 different mice and were then kept in culture with IL7 for 7 days. Afterwards, pre-B cells were transduced with either an empty vector (EV) or with BCR-ABL retroviral vectors and kept for 48 hours in +IL7 medium. Then, IL7 was removed from cells transduced with BCR-ABL1 for 1 week until cells were completely transformed. Pre-B cells transduced with EV were kept in culture with IL7 for similar culturing timepoints as transformed cells, then sorted for GFP. Total RNA of pre-B cells transduced with either EV or with BCR-ABL1 was prepared using ReliaPrep™ RNA Miniprep Kit (Promega). The total RNA library was generated using Illumina TruSeq® stranded total RNA (Gold) kit and the multiplexed samples were sequenced on Illumina HiSeq 3000 machine to produce an average of approximately 100 million paired-end reads with 150 bp in length per sample. The base calling was performed by using BCL2Fastq pipeline (version: 0.3.0) and bcl2fastq (version 2.17.1.14). PCA, Differential expression analysis and additional statistical tests related to RNA-seq were performed using R and bioconductor packages^{55,56} and in-house scripts. The broad MIT GSEA application⁵⁷ was used for Gene Set Enrichment Analysis (GSEA).

5. In Situ Proximity Ligation Assay (PLA)

For PLA experiemtns^{25,58}, the cytokine IL7 and the chemokine CXCL12 were labeled with PLA-PLUS and PLA-MINUS probes. For PLA experiments with JAK3 or p-JAK3 the corresponding antibodies were used (Cell Signaling). The PLA probes were then subjected to ligation and polymerization reactions (Sigma-Aldrich). The cells were then examined for the frequency of signals per cell under the fluorescence microscope (Leica). Pictures were taken and quantified Image J and BlobFinder software.

6. In Vivo Transplantation Of Mouse Leukemia Cells

Mouse pre-B cells from IL7Rα^{fl/fl} or FoxO1^{fl/fl} were transformed with pMIG-BCR-ABL1 (kindly provided by W. Pear) and contained either ER^{T2} or Cre-ER^{T2} were labelled with retroviral firefly luciferase and were then injected intravenously into sublethally irradiated NOD-SCID mice³⁸. Engraftment was monitored using luciferase bioimaging ³⁸. Mice were randomly allocated into each treatment group.

7. Xenografts with Human ALL Samples

NOD.Cg-Prkdcscid Il2rgtm1Wjl/SzJ (NSG) mice were purchased from Charles River and bred. All mouse housing, breeding, and surgical procedures were approved by the governmental animal care and use committees in Schleswig-Holstein (Ministerium fur Energiewende, Landwirtschaft, Umwelt, Natur and Digitalisierung). 8-12 weeks old female mice were injected intravenously with 1×10⁶ ALL cells from patient BM (>90% blasts)59,60. Animals were sacrificed upon detection of >75% leukemic blasts or clinical leukemia (loss of weight or activity, organomegaly, hind-limb paralysis). Leukemia infiltration to spleen and BM was determined⁶¹.

8. Imatinib and Antibody Treatment In Vivo

NSG mice were injected with 1×10⁶ BCR-ABL positive ALL cells/animal. 40 mg/kg of imatinib (LC Laboratories) were administered orally 5 days a week. 1 mg/kg of anti-IL7Rα antibody (clone 40131, R&D Systems) or isotype control antibody were injected intravenously on day +1, +3, +7, +21, and then every other week. Mice were sacrificed when they showed signs of leukemia or when they had at least 75% blasts in peripheral blood. Mice were randomly allocated into each treatment group and no blinding was used.

9. Flow Cytometry

Antibodies for flow cytometry (CD19, IL7R, CXCR4, FoxO1 and CD11b) were purchased from (eBiosciences, BioLegend, Invitrogen or Cell Signaling). Intracellular flow cytometry staining was performed using Fix and Perm cell permeabilization kit (ADG). Cell viability was measured using Sytox® blue dead cell stain (Life Technologies). FACS Cantoll (BD Biosciences) was used for flow cytometry, and FlowJo v.10.1 was used for data analysis.

10. Measurement of Ca²⁺ flux

A total of 1×10⁶ cells were loaded with Indo-1 AM (Invitrogen) and used for Ca²⁺ analyses⁶². 100 ng/m1 of CXCL12 was used for stimulation

11. Western Blot

Wet-western blotting was performed⁶¹. P-JAK3, P-JAK2, P-JAK1, P-STATS, P-FoxO1, JAK1, JAK2, JAK3, STATS, FoxO1 and GAPDH antibodies were obtained from Cell Signaling Technology.

12. Statistical Analysis

Statistical tests are indicated in the figure legends. Results were analyzed for statistical significance with GraphPad Prism 8.3.0 software or SPSS (v 24.0.0.2). A p value of <0.0500 was considered significant (*p<0.005, **p <0.001, ***p <0.001, ****p <0.001). In vitro panels are representative of at least 3 independent experiments, unless mentioned otherwise.

13. Ruxolitinib, Imatinib and Antibody Treatment In Vivo

NSG mice were injected with 1×10⁶ SUP-B15 BCR-ABL positive ALL cells/animal. 60 mg/kg of ruxolitinib (LC LAboratories) or 40 mg/kg of imatinib (LC Laboratories) or combination of both inhibitors were administered orally 5 days a week. Mice were sacrificed when they showed signs of leukemia. Mice were randomly allocated into each treatment group and no blinding was used.

14. Cell Cycle Analysis

For cell cycle analyses, Click-iT® EdU Alexa Fluor® 647 Imaging Kit (Invitrogen) or BrdU Flow Kit (BD Biosciences) were used.

15. Colony Formation Assay

1×10⁵ CXCR4^{fl/fl} mbl Cre-ERT2 cells transformed with pMIG-BCR ABL1 were treated with either ethanol or tamoxifen and then used for colony formation assay as described previously (Miller et al., Methods Mol Biol 290, 71-89 (2005)).

16. Chemotaxis

Chemotaxis assay was performed as described (Calpe, E., et al. Blood 118, 4401-4410 (2011)). 5×10⁵ cells were seeded on the top chamber of a transwell culture insert (Corning) and allowed to migrate toward media containing 100 ng/ml CXCL12 (ImmunoTools) for 16 h. The cell number in the lower chamber was determined with hematocytometer.

17. Immunoprecipitation

SUP-B15 Ph+ ALL cells were lysed and an Immunoprecipitation (IP; DynabeadsTM Protein A Immunoprecipitation kit; Thermoscientific) was performed using an antibody against the breakpoint cluster region protein (BCR; SantaCruz), or IgG2a (isotype; Southern Biotech). The proteins from the IP were used for western blotting to detect the presence of IL7R and CXCR4 proteins (R&D, and Invitrogen, respectively).

### B. Example 2

### BCR-ABL1 Alters the Expression of IL7R and CXCR4 Regulated Genes

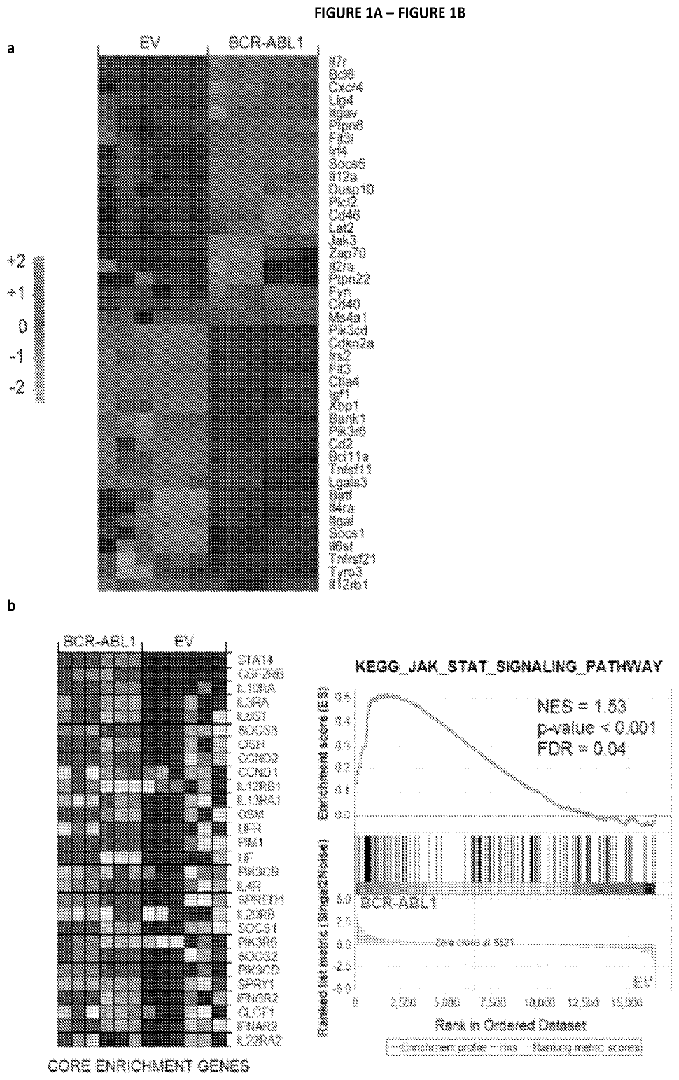

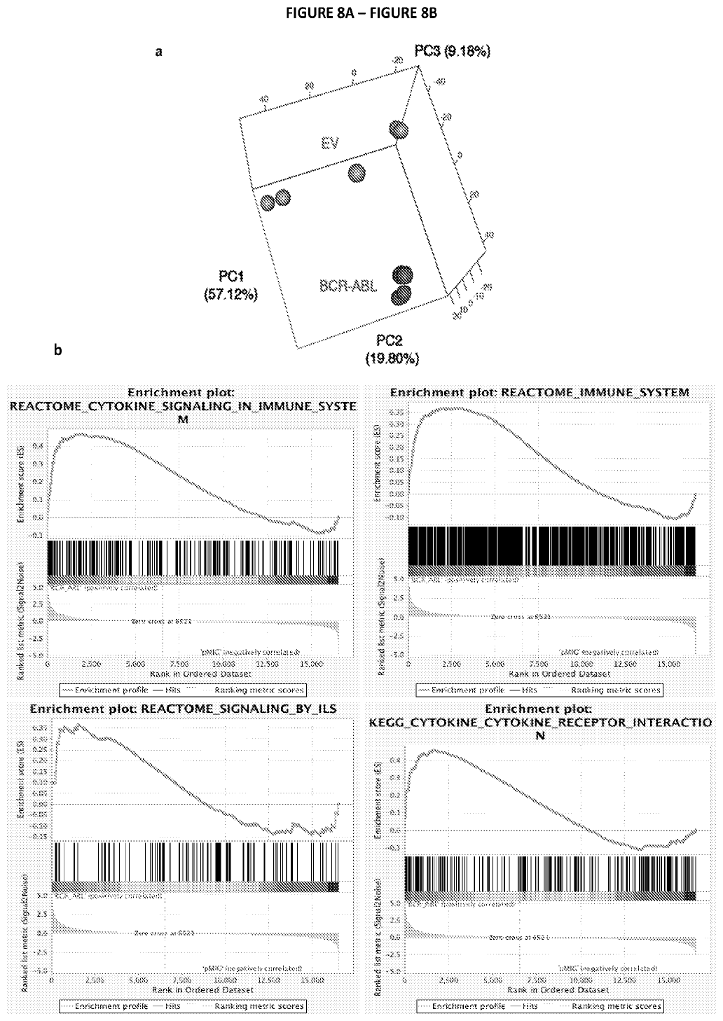

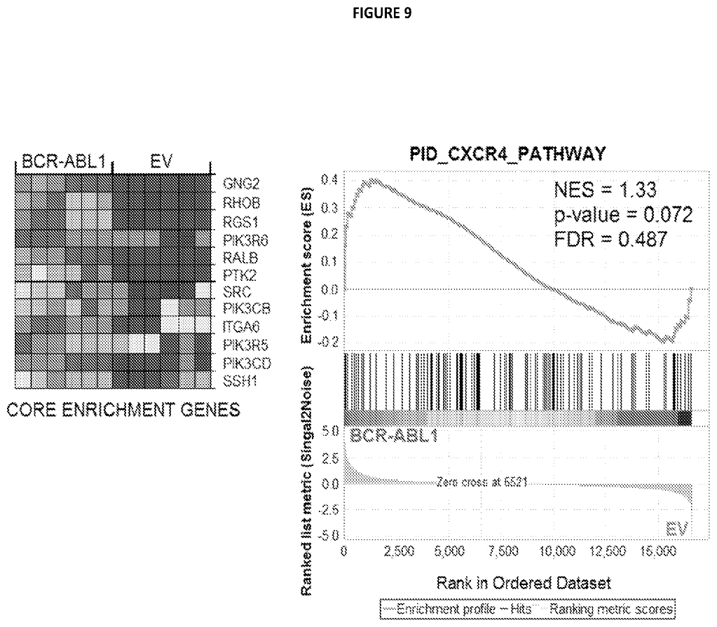

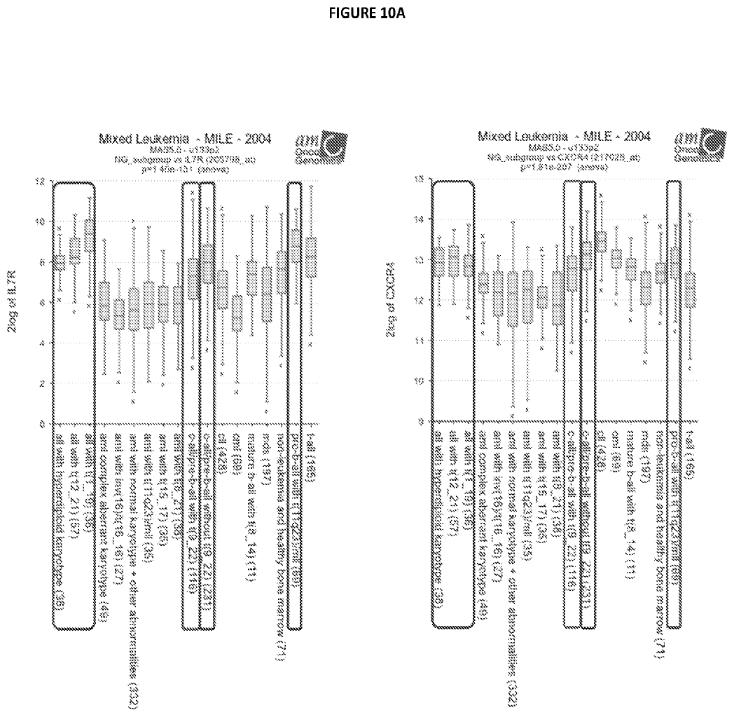

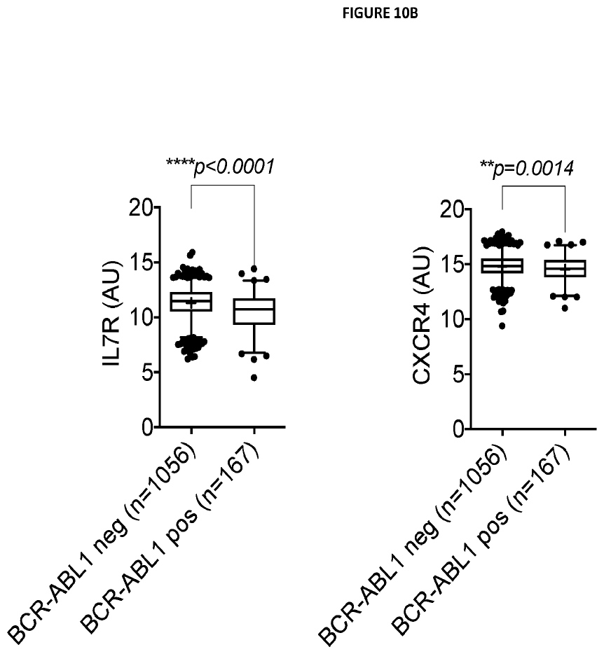

To better understand the molecular mechanisms regulating BCR-ABL1 induced transformation and the development of Ph+ ALL, we performed RNA-sequencing (RNA-Seq) and compared transcriptome profile of transformed cells to wildtype (WT) pre-B cells. To this end, 6 individually generated control WT pre-B cell lines and 6 BCR-ABL-transformed pre-B cell counterparts were analyzed. Global transcription profile based Principal Component Analysis (PCA) showed clear segregation of WT and BCR-ABL1-transformed cells (cumulative explained variance=86.1%; Supplementary FIG. 1a). Gene Ontology (GO) analysis for biological processes (BP) was performed and genes that were differentially regulated between the two groups were further investigated, particularly genes related to lymphocyte activation, proliferation and migration (FIG. 1a). The analysis showed a differential regulation in multiple signaling pathways related to IL7R signaling (FIG. 1a and FIG. 8a). To assess the importance of IL7R signaling related pathways and processes in BCR-ABL1, we performed Gene Set Enrichment Atanalysis (GSEA) on IL7R related KEGG and REACTOME MSigDB genesets (Broad Institute, Inc., Massachusetts Institute of Technology (MIT), and Regents of the University of California). Of the 8 genesets analyzed, 5 showed statistically significant upregulation in BCR-ABL1 as compared to control samples (False Discovery Rate (FDR) <0.25; FIG. 1b, FIG. 8a). Interestingly, genes involved in JAK/STAT signaling, signaling by interleukins, and cytokine signaling were among the most significantly altered genesets (FIG. 1b, FIG. 8b). CXCR4 pathway, though not statistically significant, showed positive correlation to the BCR-ABL1 transformed samples (FIG. 9). In addition, the expression of several cytokine signaling regulators such as the transcription repressor BCL6 as well as several phosphatases including PTPN6, PTPN22 and DUSP10 were deregulated by BCR-ABL1 (FIG. 1a). In this work we focused in the role of IL7R and CXCR4.

### C. Example 3

### IL7 Rescues BCR-ABL1 Transformed Cells from Inhibitor Treatment

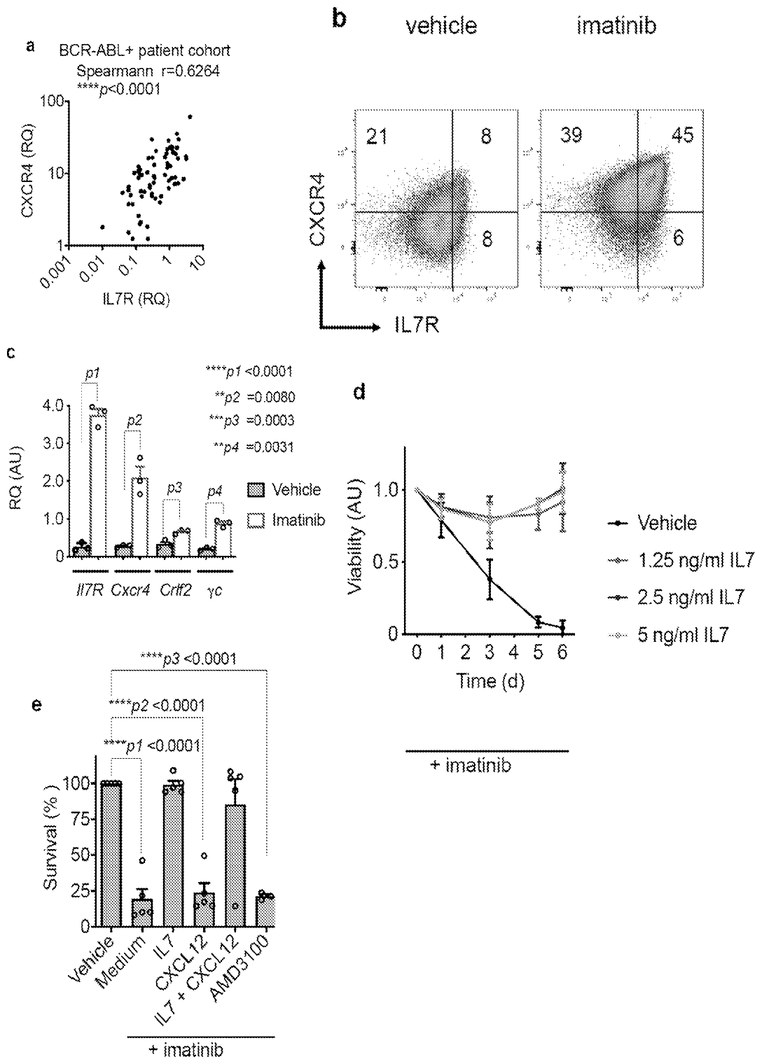

Our data suggest that the signaling pathways of IL7R and CXCR4 are tightly regulated by the activity of the oncogenic kinase BCR-ABL1 and therefore we hypothesized that they might be directly involved in malignant transformation. To test whether the expression of IL7R and CXCR4 is also correlated in primary ALL, we analyzed a cohort of 68 Ph⁺ BCP-ALL patients and found significant correlation of IL7R and CXCR4 gene expression (Spearman r=0.6264; p<0.0001; FIG. 2a) suggesting that our sequence analysis of BCR-ABL1 transformed pre-B cells is in accordance with in vivo condition. In addition, searching in a mixed leukemia gene expression study¹⁹ using the R2 database (http://r2.amc.nl) showed that IL7R and CXCR4 are expressed at reduced levels in BCR-ABL+ ALL (t9; 22) in comparison to other BCP-ALL entities (FIG. 10a). Similar results were also observed in RNA-sequencing dataset of 1,223 BCP-ALL patients²⁰ (FIG. 10b).

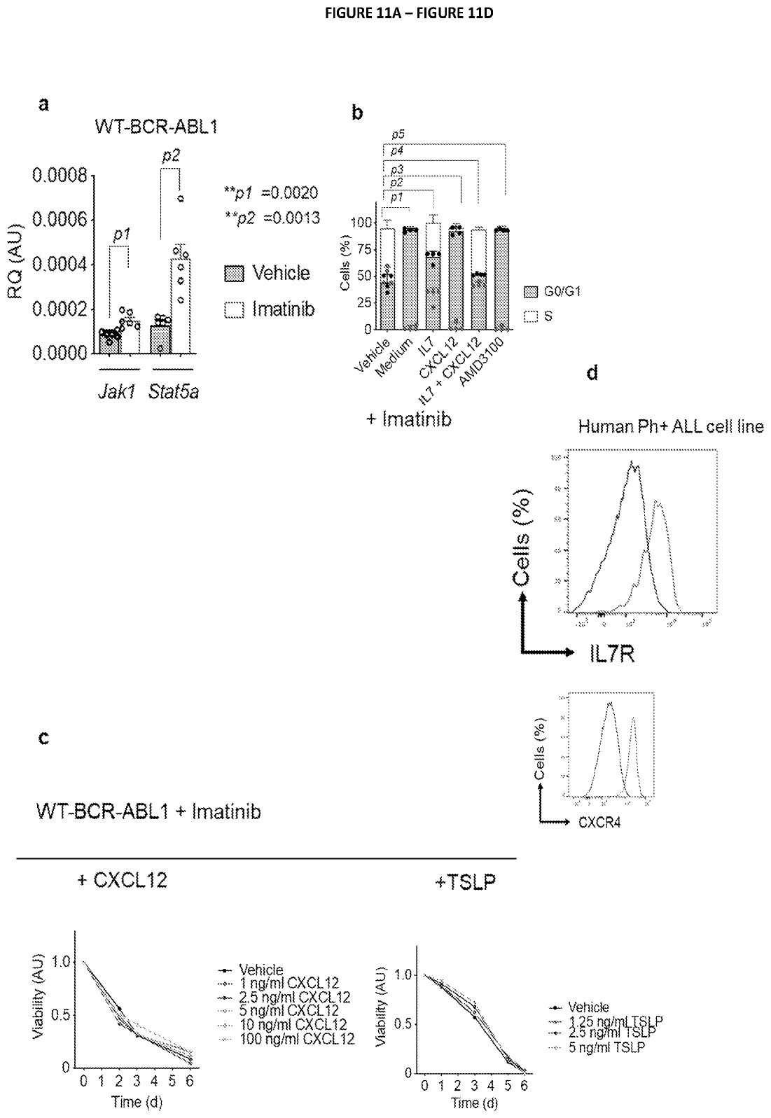

Interestingly, the inhibition of BCR-ABL1 kinase by imatinib treatment resulted in an upregulated expression of both the chemokine receptor CXCR4 and the IL7R together with downstream signaling elements such as Jakl and STAT5a (FIG. 2b-c, and FIG. 11a). To test whether the upregulation of IL7R or CXCR4 expression upon BCR-ABL1 kinase inhibition affects the survival of BCR-ABL1 transformed cells, we investigated imatinib-induced cell death in the presence of the respective cytokine/chemokine. We found that treatment with IL7 counteracted imatinib-induced cell death and restored the cell cycle progression (FIG. 2d and FIG. 10b). However, treatment with CXCL12, the ligand for CXCR4, or its antagonist AMD3100 did not affect imatinib treatment (FIG. 2e and FIG. 11b-c). Likewise, treatment with TSLP, the ligand for CRLF2, was also unable to rescue BCR-ABL1 transformed cells from inhibitor-induced cell death (FIG. 11c). Interestingly, human Ph+ ALL SUP-15 cells also upregulated IL7R and CXCR4 in response to imatinib treatment (FIG. 11d). Together, these data suggest that Ph+ ALL cells upregulate growth factor receptors including IL7R which might enable the survival of Ph⁺ cells in microenvironments containing IL7 despite ABL1 kinase inhibitor treatment.

### D. Example 4

### BCR-ABL1 Transformation Requires IL7R Expression

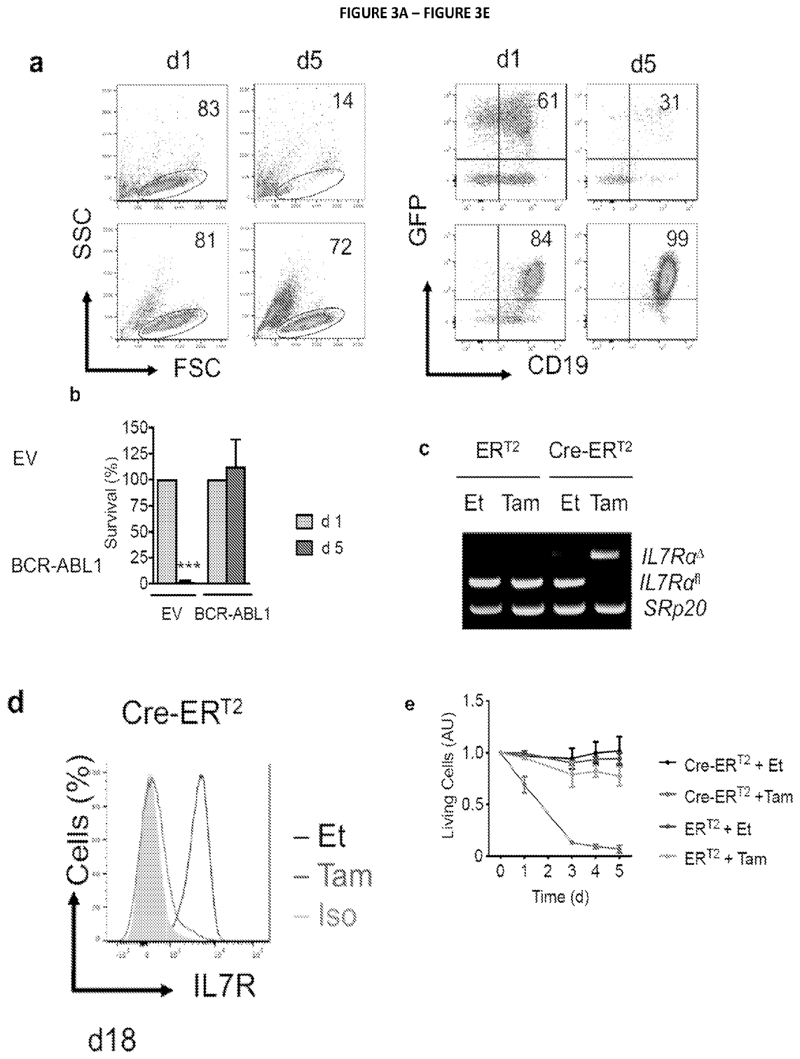

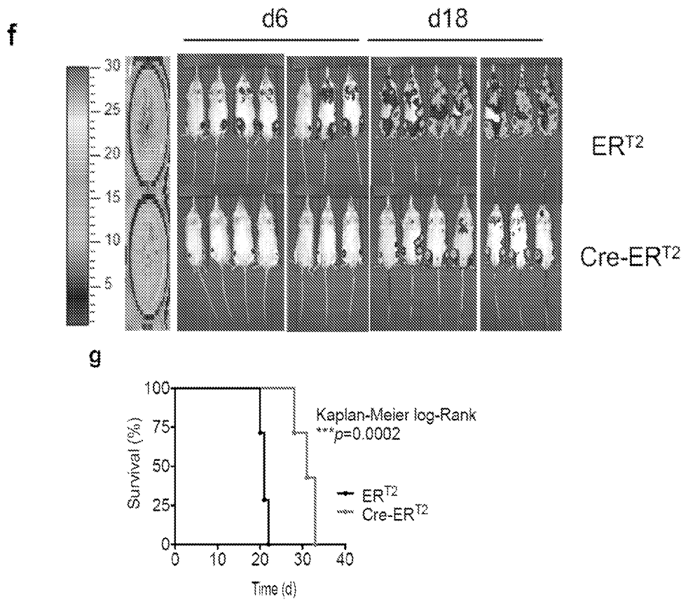

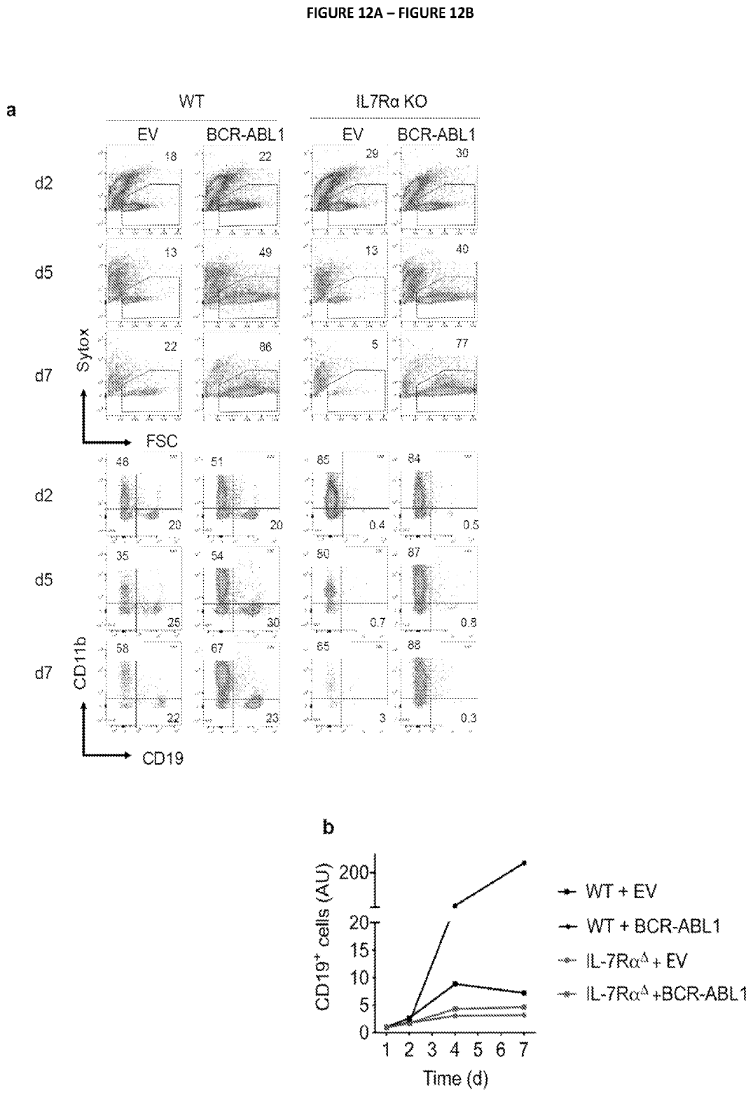

The upregulation of IL7R under imatinib treatment raised the question whether IL7R expression is required for BCR-ABL1 induced pre-B cell transformation and ALL development. Therefore, we generated BCR-ABL1 transformed bone marrow (BM)-derived pre-B cells from mice homozygous for loxP-flanked IL7Ra alleles (IL7Raf^{fl/fl})²¹. Usually, pre-B cells proliferate in the presence of growth factors such as IL7. However, the expression of BCR-ABL1 results in growth factor-independent proliferation in the absence of IL7 (FIG. 3a-b). For inducible deletion of the IL7Ra gene, we introduced a tamoxifen-inducible Cre (Cre-ER^{T2}) into the BCR-ABL1-transformed IL7Rα^{fl/fl} cells. Inducible deletion of the IL7Rα gene led to cell death of the BCR-ABL1-transformed pre-B cells (FIG. 3c-e). To determine the role of IL7R expression in vivo, we injected BCR-ABL1-transformed IL7Rα^{fl/fl} pre-B cells into NOD-SCID immunodeficient recipient mice. IL7R deletion by tamoxifen treatment in vivo reduced leukemic cell burden and significantly prolonged the survival of xenograft mice injected with BCR-ABL1 transformed cells (FIG. 3f-g). In support of these results, BM-derived cells from IL7Rα-deficient mice²² did not give rise to BCR-ABL1 transformed pre-B cells, while BCR-ABL1 transformed myeloid cells (CD11b⁺) can readily be generated from the same cells (FIG. 12). Together, our data suggest that IL7R expression is specifically required for the initiation and the maintenance of pre-B cell transformation and ALL development.

### E. Example 5

### IL7R Synergizes with CXCR4 for BCR-ABL1 Induced Transformation

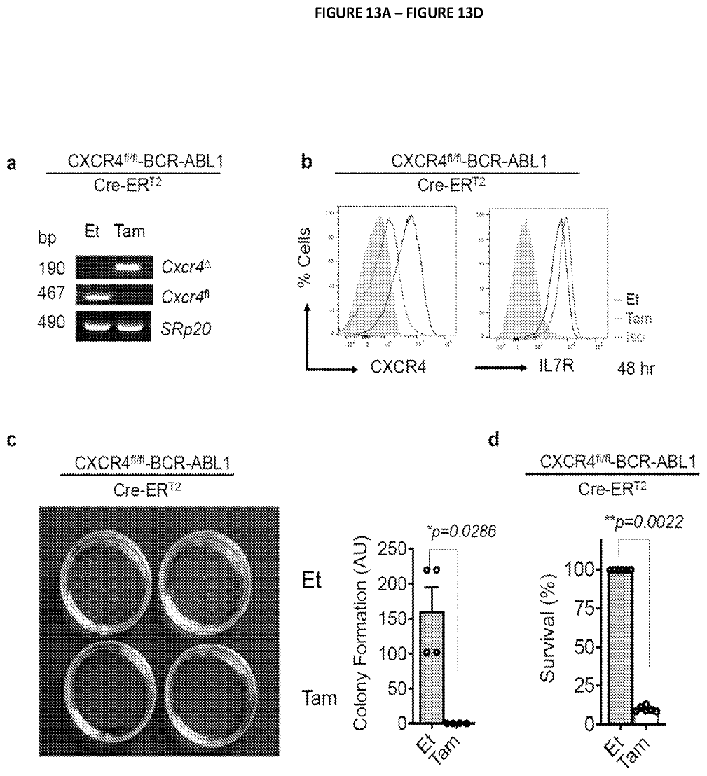

Since CXCR4 regulated genes were also altered, we tested whether CXCR4 is also required for BCR-ABL1 induced transformation. Therefore, we generated BCR-ABL1 transformed pre-B cells from mice homozygous for CXCR4 loxP-flanked alleles²³ (CXCR4^{fl/fl}). Deleting CXCR4 in these cells using Cre-ER^{T2} resulted in rapid cell death and inability of BCR-ABL1 cells to form colonies in vitro (FIG. 13).

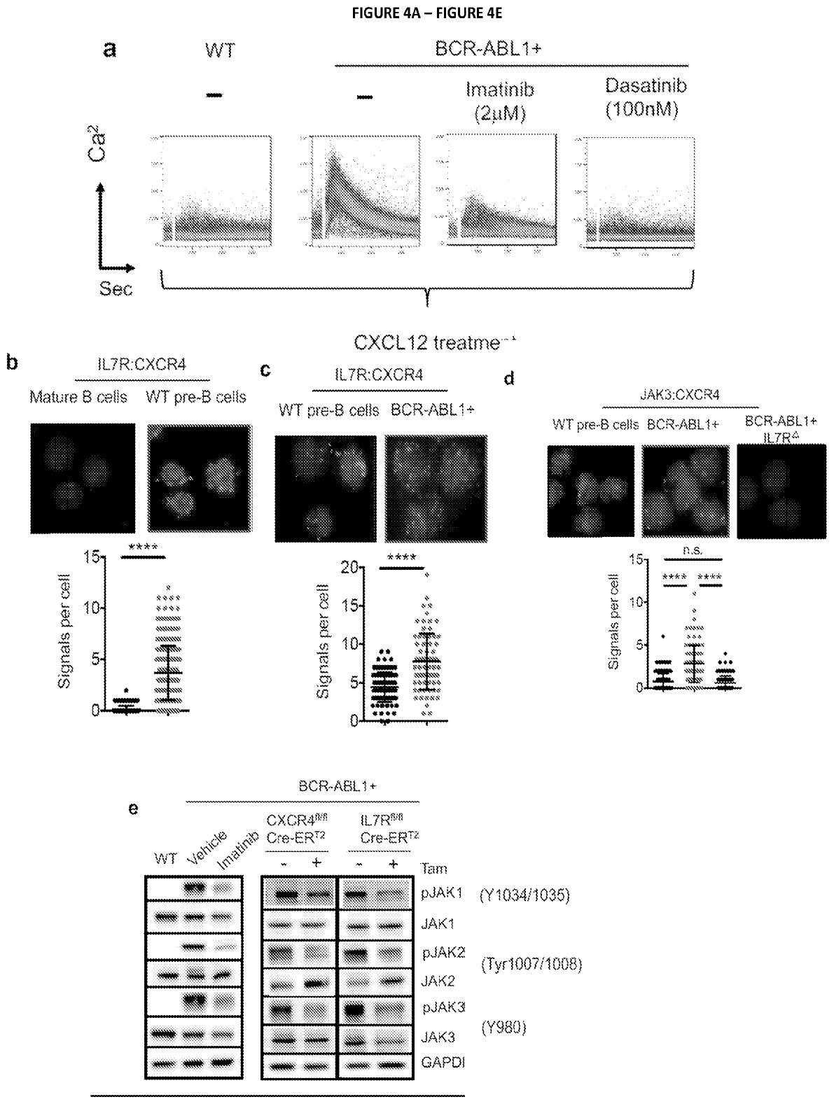

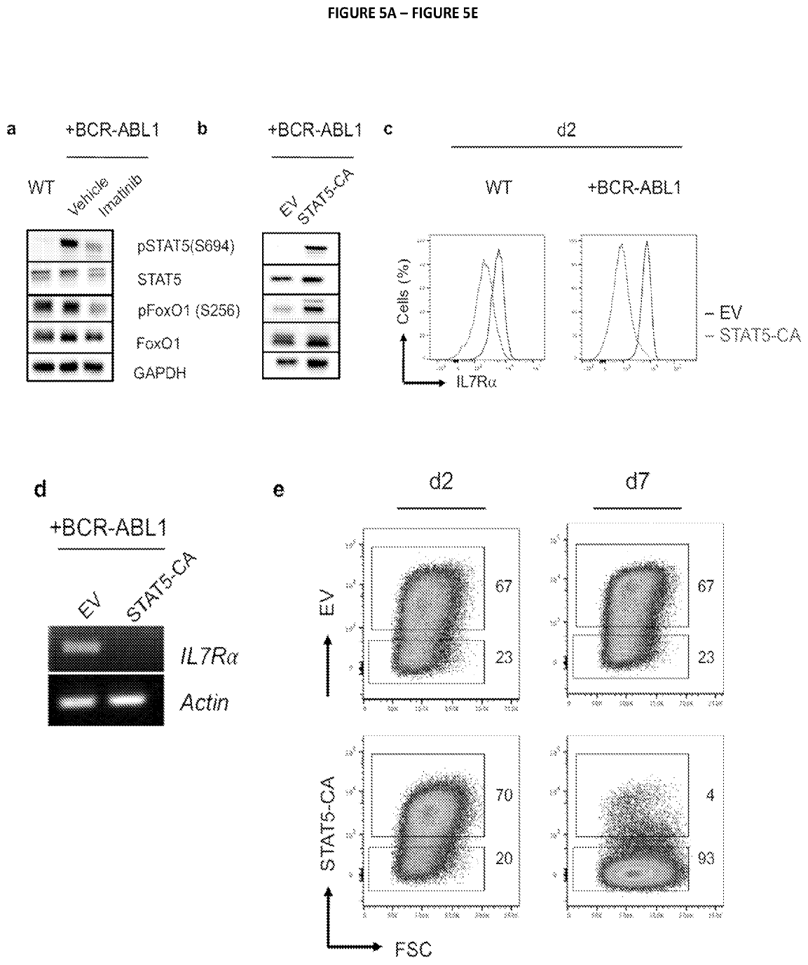

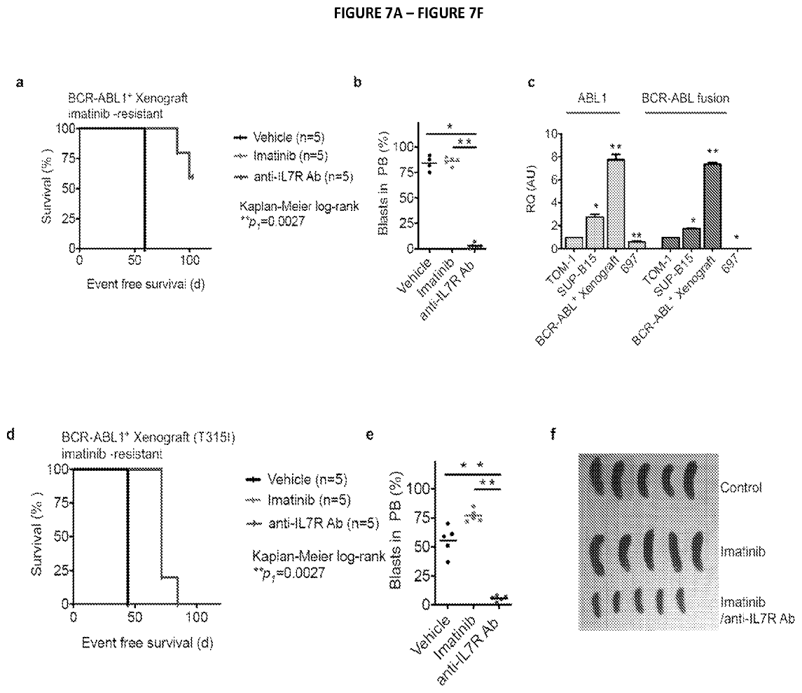

Since BCR-ABL1 was reported to be involved in crosstalk with CXCR4²⁴, we investigated whether the requirement for IL7R and CXCR4 in BCR-ABL1 induced transformation is mediated by spatial receptor colocalization. We first examined the effect of BCR-ABL1 recruitment on CXCR4-mediated Ca²⁺ mobilization²⁵. Therefore, the CXCL12-induced Ca²⁺ flux in WT as compared with BCR-ABL1 transformed cells was tested. While WT cells showed a negligible CXCL12-induced Ca²⁺ flux, BCR-ABL1 transformed cells showed a robust Ca²⁺ response (FIG. 4a). Inhibiting the BCR-ABL1 kinase activity by either imatinib or dasatinib blocked the CXCL12-induced Ca²⁺ response with dasatinib showing an effective inhibition at much lower concentrations than imatinib, which is most likely caused by the additional effect of dasatinib on Src kinases²⁶ (FIG. 4a). Importantly, inducible deletion of CXCR4 in BCR-ABL1 transformed cells or treating them with AMD3100, an antagonist of CXCL12, prevented the Ca²⁺ response (Supplementary FIG. 7a). These data are in full agreement with the view that BCR-ABL1 is recruited to CXCR4 and can be activated by the respective ligand CXCL12.

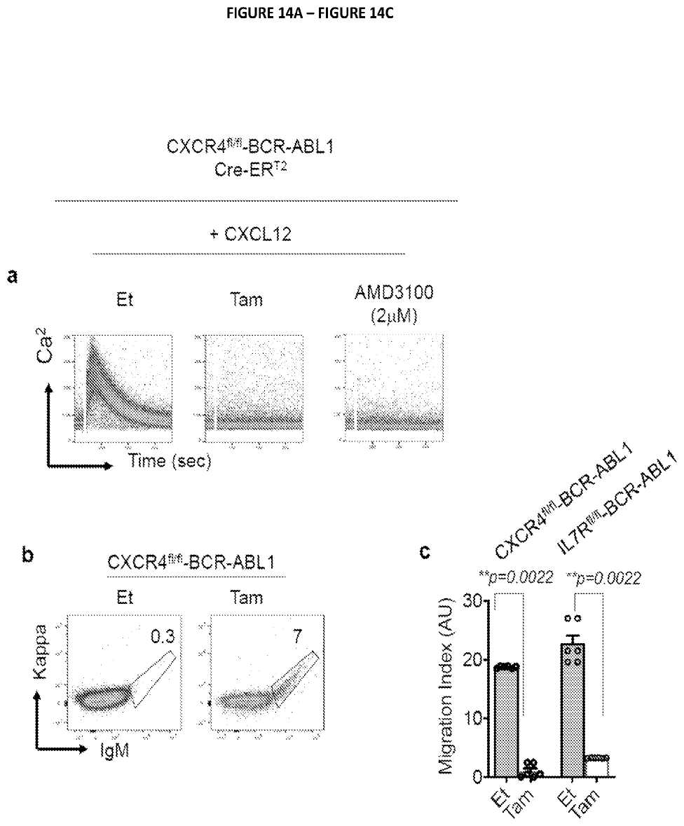

Interestingly, the CXCR4-deficient pre-B cells showed an increased differentiation capacity as measured by the elevated ratio of cells expressing the immunoglobulin kappa light chain (FIG. 14b). These data suggest that CXCR4 cooperates with IL7R in preventing pre-B cell differentiation²⁷. Similarly, IL7R seems to act together with CXCR4 in directing cell migration, as both CXCR4-defcient and IL7R-defcient BCR-ABL1-transformed cells show an impaired migration towards a CXCL12 gradient (FIG. 14c).

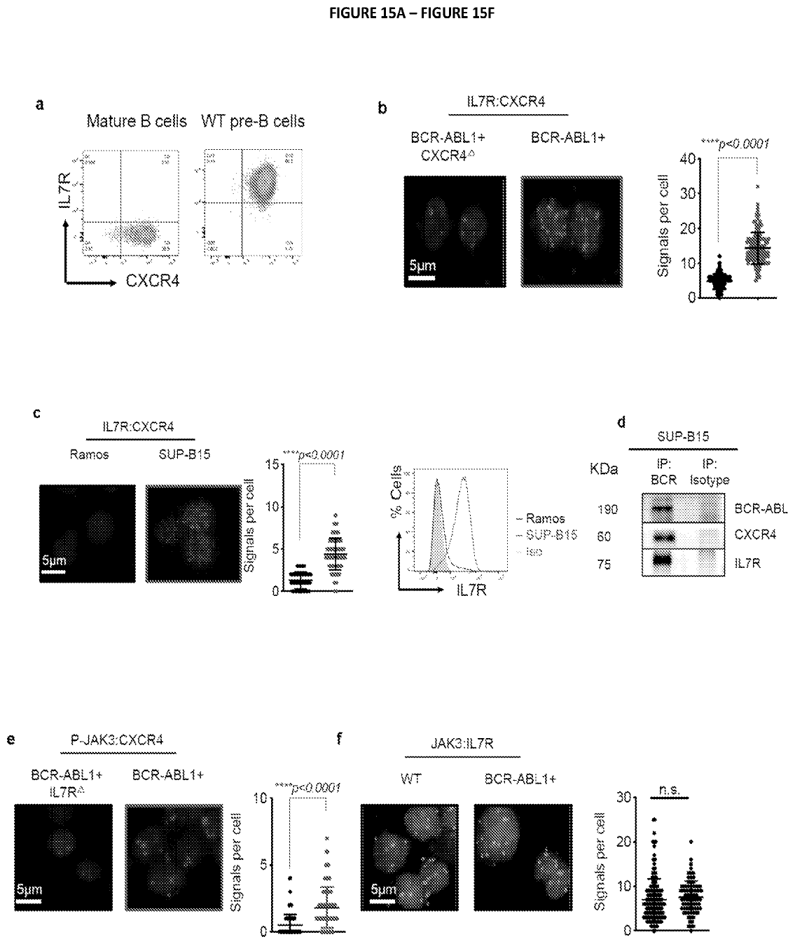

To study further how IL7R and CXCR4 act synergistically to regulate pre-B cell differentiation and proliferation, we investigated the interaction between IL7R and CXCR4 by proximity ligation assay (PLA). Adjacent binding of the ligands IL7 (7 kD) and CXCL12 (15 kD) suggests that the corresponding receptors are localized on the cell surface at a proximity below l0nm in precursor B cells (FIG. 4b and FIG. 15a). Interestingly, BCR-ABL1 transformed pre-B cells show an increased number of IL7R/CXCR4 foci as compared with untransformed WT pre-B cells (FIG. 4c and FIG. 15b). This association is also detected in human BCR-ABL+ pre-B ALL cells (FIG. 15c). The interaction between CXCR4/IL7R and BCR-ABL was further confirmed by immunoprecipitation (FIG. 15d). Together, these findings suggest that the interaction between IL7R and CXCR4 is increased in BCR-ABL1 transformed pre-B cells. Hence, we postulated that this interaction recruits IL7R-associated signaling proteins into close proximity to CXCR4 thereby enabling activation by BCR-ABL1 which then leads to pre-B cell transformation. To directly test this hypothesis, we investigated the association of Jak3 with CXCR4 in BCR-ABL1 transformed cells as compared to WT control. Usually, Jak3 is associated with the yc subunit of IL7R but not with CXCR4. In fact, CXCL12/CXCR4 signaling was shown to be independent of JAK3²⁸. Interestingly, a significant IL7R-dependent increase in Jak3 association with CXCR4 was observed in BCR-ABL1 transformed pre-B cells (FIG. 4d). Similarly, an IL7R-dependent association of CXCR4 with p-Jak3 was observed in BCR-ABL1 transformed cells (FIG. 15e). In contrast, the association between IL7R and Jak3 showed no significant change suggesting that BCR-ABL1 induced transformation has no effect on IL7R interaction with its downstream signaling elements (FIG. 150.

As expected, Jak kinases show increased phosphorylation in BCR-ABL1 transformed cells and imatinib treatment reduces this phosphorylation (FIG. 4e). In full agreement with the hypothesis that the interaction between CXCR4 and IL7R enables CXCR4 to utilize the downstream signaling machinery of IL7R, inducible inactivation of either IL7R or CXCR4 expression results in decreased activity of Jakl, Jak2 and Jak3 as shown by their reduced phosphorylation (FIG. 4e).

Together, these data suggest that BCR-ABL1 interaction with CXCR4 recruits this oncogene into the proximity of IL7R-associated Jak kinases thereby enabling their BCR-ABL1 mediated activation and pre-B cell transformation.

### F. Example 6

### BCR-ABL1 Controls IL7R Expression by Regulating Fox01

Activated JAK kinases phosphorylate the cytoplasmic domain of cytokine receptors at specific tyrosine residues leading to the recruitment and subsequent activation of STAT (Signal Transducer and Activator of Transcription) proteins. Phosphorylated STATs undergo dimerization and translocate to the nucleus where they activate target genes involved in proliferation and survival of lymphocytes²⁹. As expected, increased STAT5 phosphorylation was detected in BCR-ABL1 transformed cells and ABL1 kinase activity was required for this increase (FIG. 5a). Since STAT5 is activated by IL7R signaling³⁰, and also by BCR-ABL1, we postulated that activated STAT5 might control IL7R expression in a negative feedback loop that prevents deregulated IL7R expression. The transcription factor FoxO1 was shown to regulate the expression of IL7R¹² as well as CXCR4^{31,32}. The fact that STAT5 activates PI3K signaling³³ which in turn suppresses FoxO1 transcriptional activity by phosphorylation of specific S/T sites, suggests that STAT5 activation can lead to increased FoxO1 phosphorylation and subsequent downregulation of IL7Ra expression. Indeed, imatinib treatment of BCR-ABL1 transformed pre-B cells resulted in deacreased FoxO1 phosphorylation (FIG. 5a). Moreover, introducing a constitutively active STAT5 version (STAT5-CA)³⁴ into pre-B cells resulted in FoxO1 inactivation, as measured by increased 5256 phosphorylation, and decreased IL7Rα expression (FIG. 5b-c). In full agreement of transcriptional repression, reverse transcriptase PCR experiments revealed that IL7Ra transcripts were almost missing in STAT5-CA expressing cells (FIG. 5d). Interestingly, the reduced IL7R expression was associated with loss of the cells expressing STAT5-CA (FIG. 5e). These findings suggest that STAT5 regulates IL7R expression in a negative feed-backloop and that fine-tuned STAT5 activity in transformed cells is important to induce cell proliferation and, at the same time, avoid destruction of IL7R expression by excessive STAT5 activity. Altogether, we propose that BCR-ABL1 controls IL7R expression by activating a common STAT5-regulated negative feedback mechanism and that the obsereved downregulation of IL7R expression by BCR-ABL1 guarantees a fine-tuned STAT5 activity.

### G. Example 7

### Fox01 is Required for Leukemogenesis

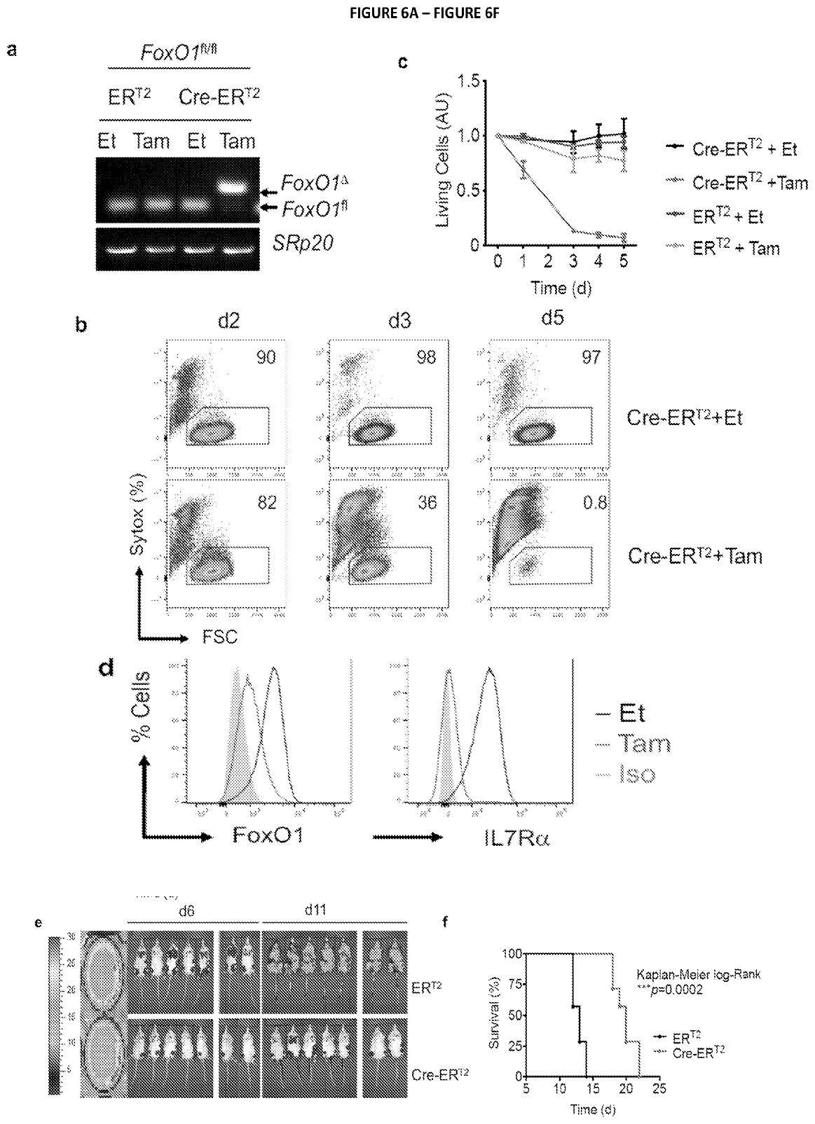

To further confirm the requirement of FoxO1 transcription factor for BCR-ABL1 mediated leukemogenesis, we generated BCR-ABL1-transformed pre-B cells from mice homozygous for loxP-flanked alleles of FoxO1 (FoxO1^{fl/fl}). To induce FoxO1 deletion, we introduced into the BCR-ABL1-transformed cells our tamoxifen-inducible Cre-ER^{T2} by retroviral transduction. Inducible deletion of FoxO1 led to cell loss of the BCR-ABL1-transformed cells (FIG. 6a-c). Moreover, deletion of FoxOl resulted in concomitant downregulation of FoxO1 and IL7R expression (FIG. 6d). To provide further evidence for the important role of FoxO1 in leukemogenesis in vivo, we injected BCR-ABL1-transformed FoxO1^{fl/fl} cells into sub-lethally irradiated NOD/SCID recipient mice and monitored development of leukemia after deletion of FoxO1 as compared with controls in vivo. We found that BCR-ABL1-transformed FoxO1^{fl/fl} caused fatal leukemia within 2 weeks, while deleting FoxOl by tamoxifen-induced Cre-ER^{T2} activation reduced the leukemic cell burden and prolonged the survival time of respective mice (FIG. 6e-f).

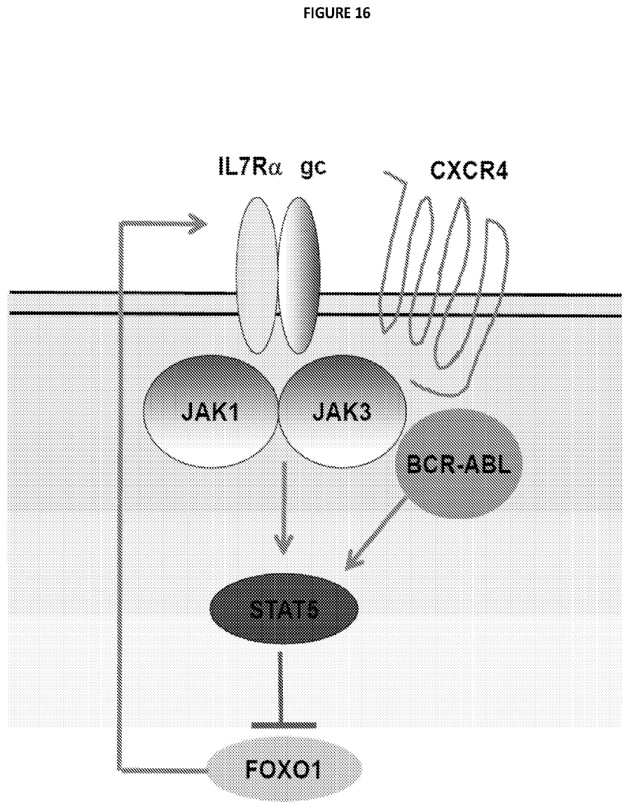

Together, these data suggest that FoxO1 plays an essential role in BCR-ABL1-induced transformation most likely through the activation of IL7R expression (FIG. 16).

### H. Example 8

### Blocking IL7R Prevents Leukemia Development of BCR-ABL1+ Cells

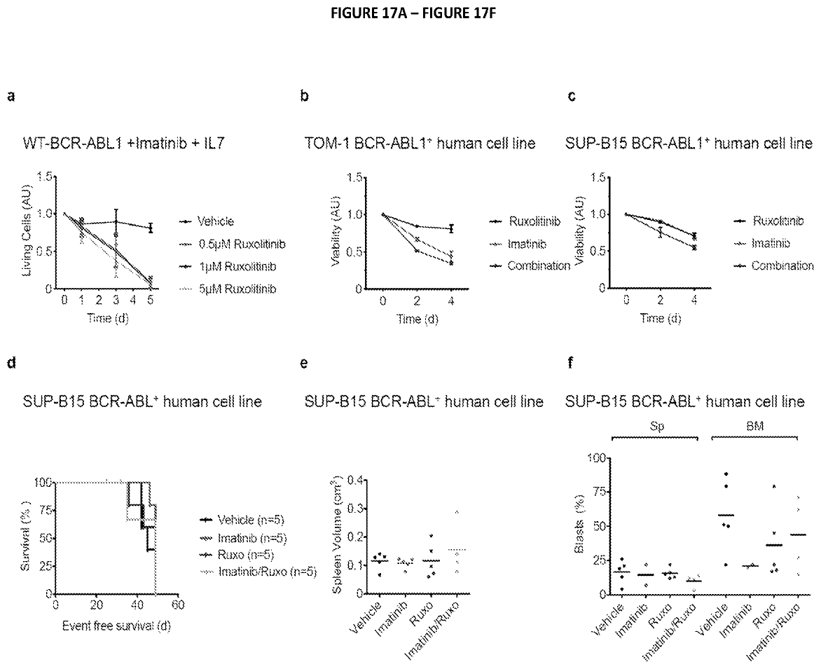

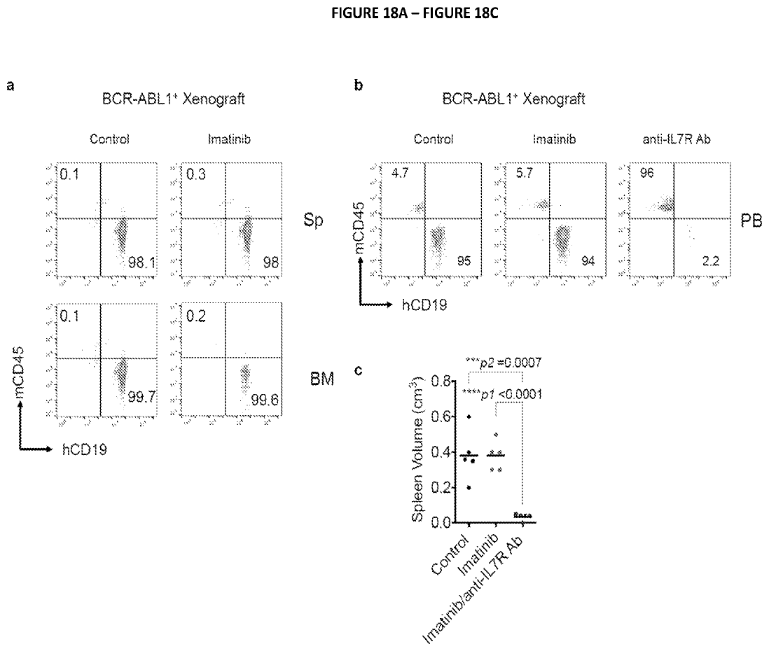

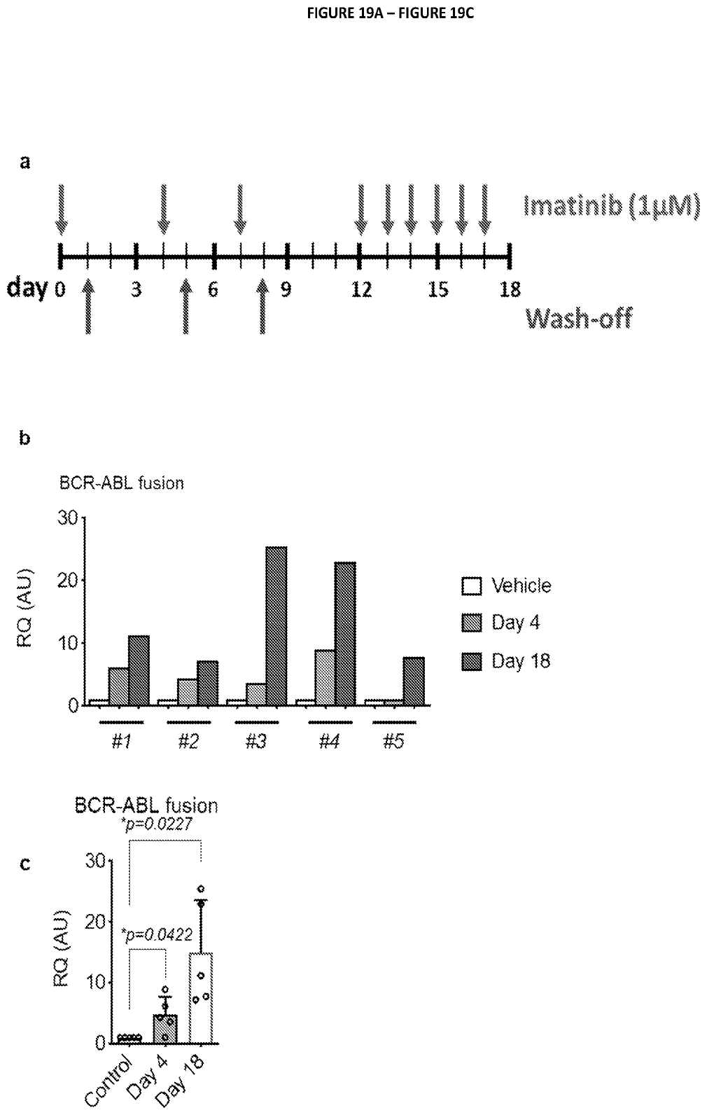

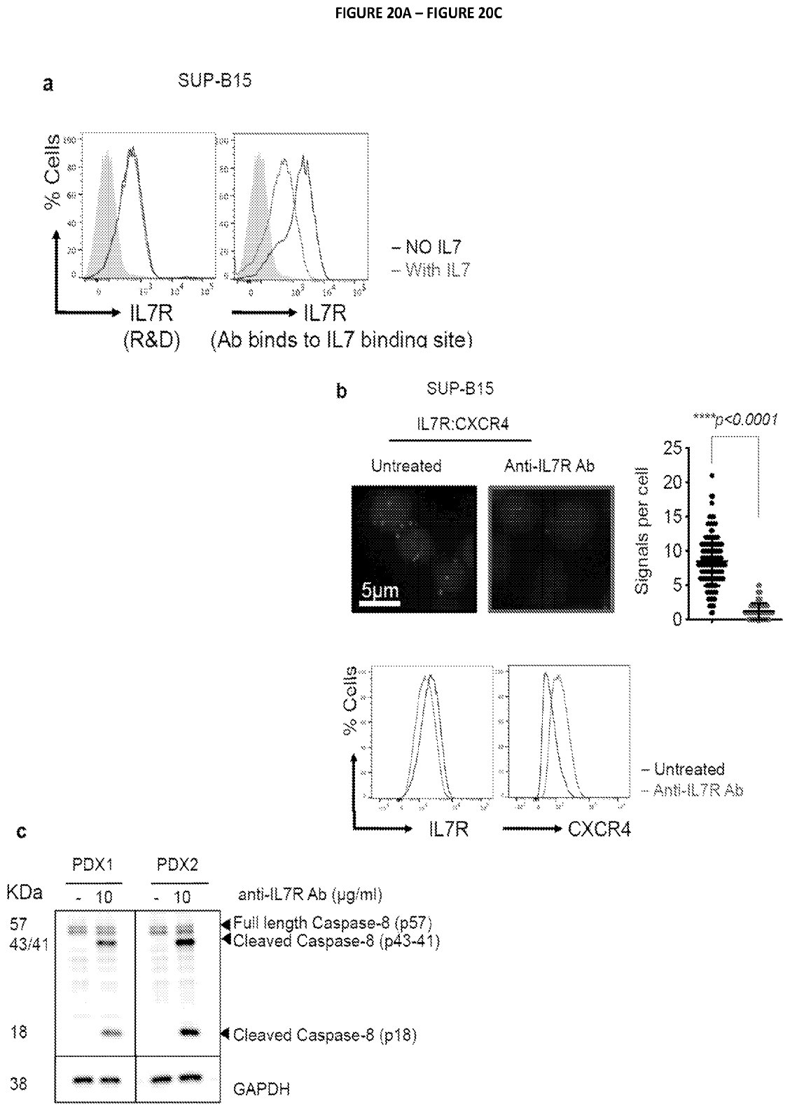

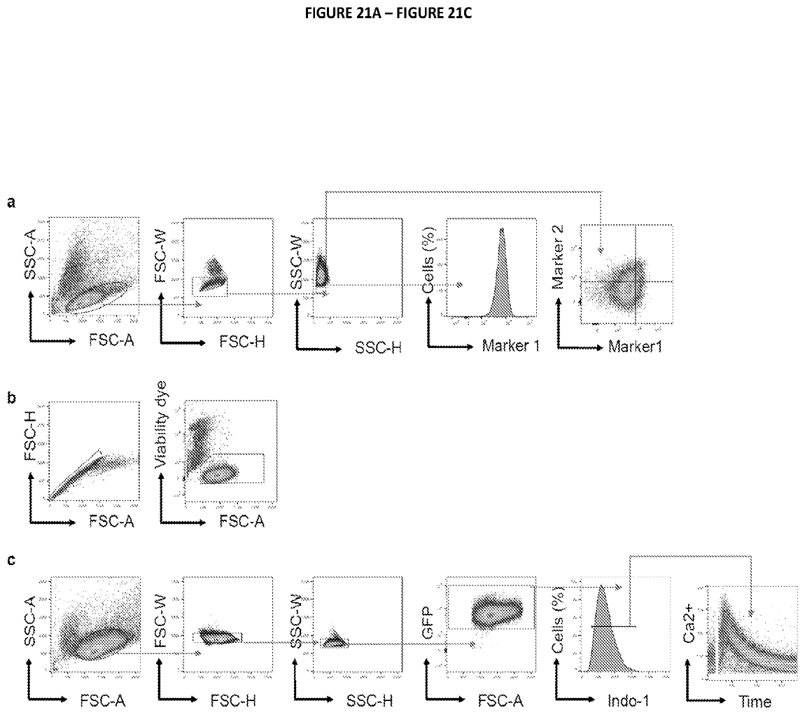

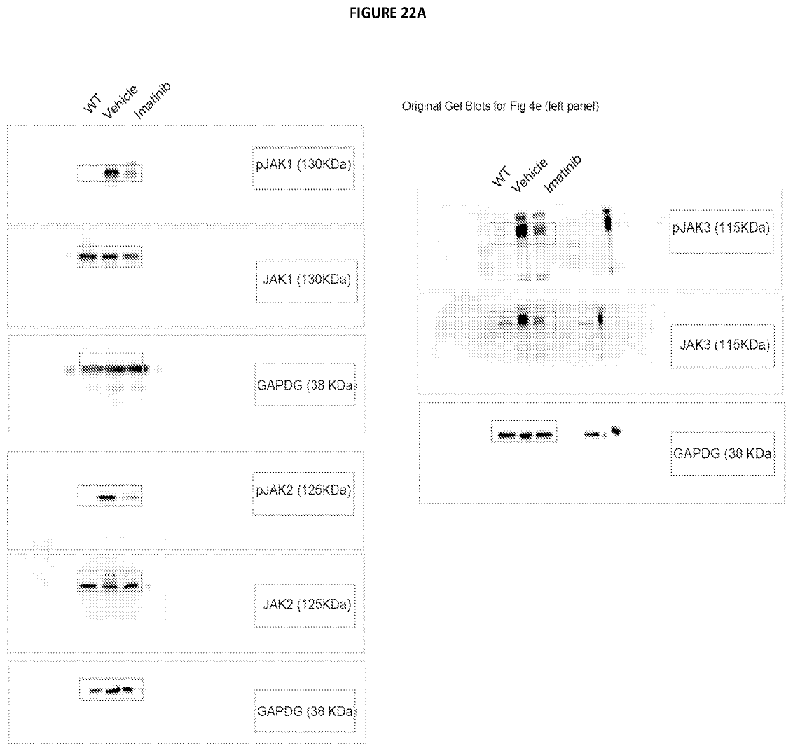

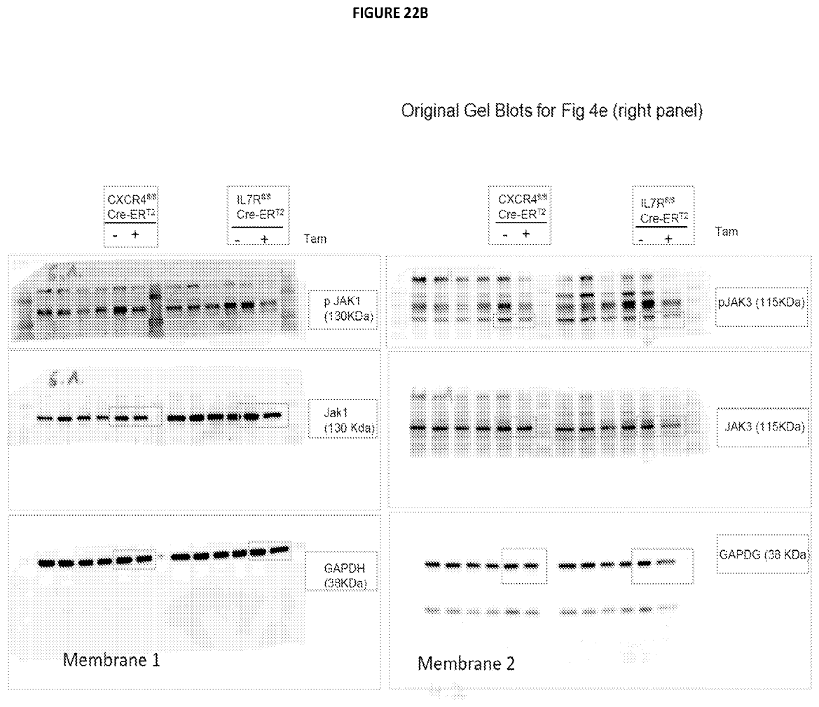

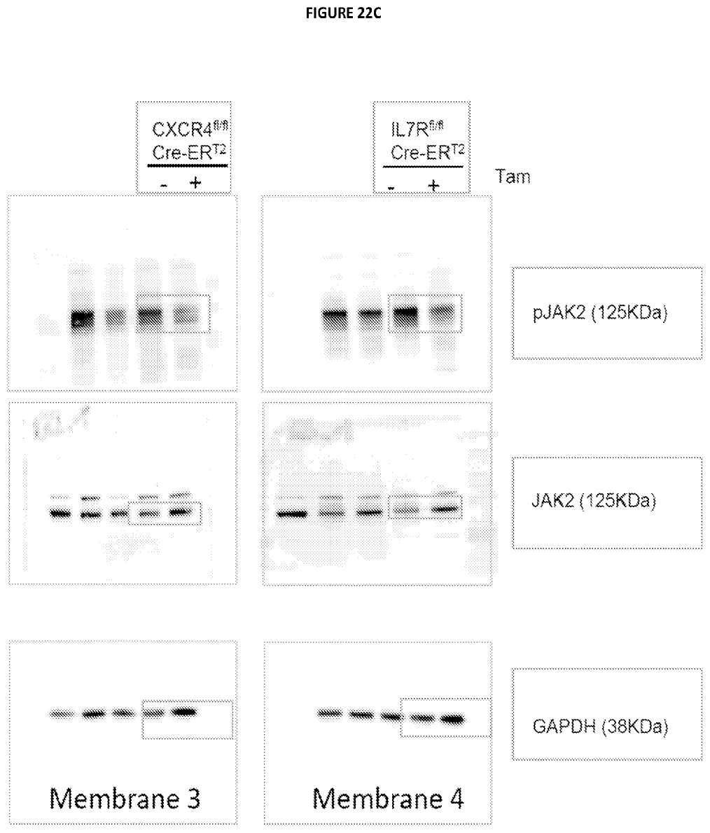

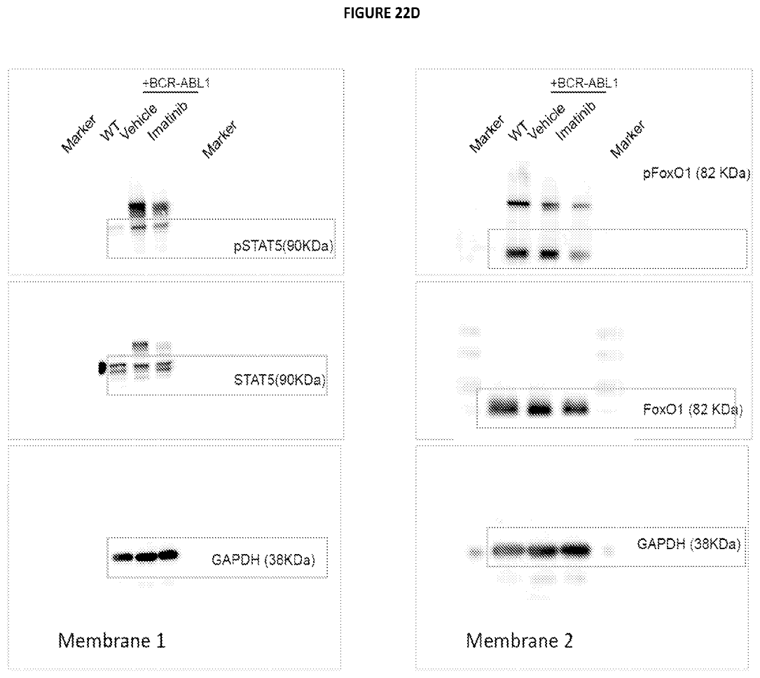

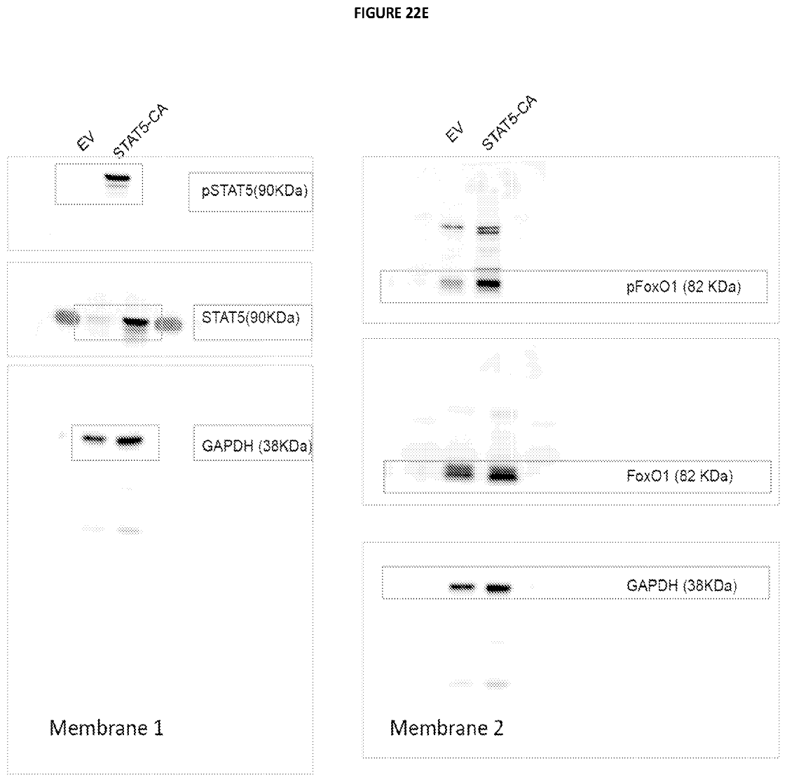

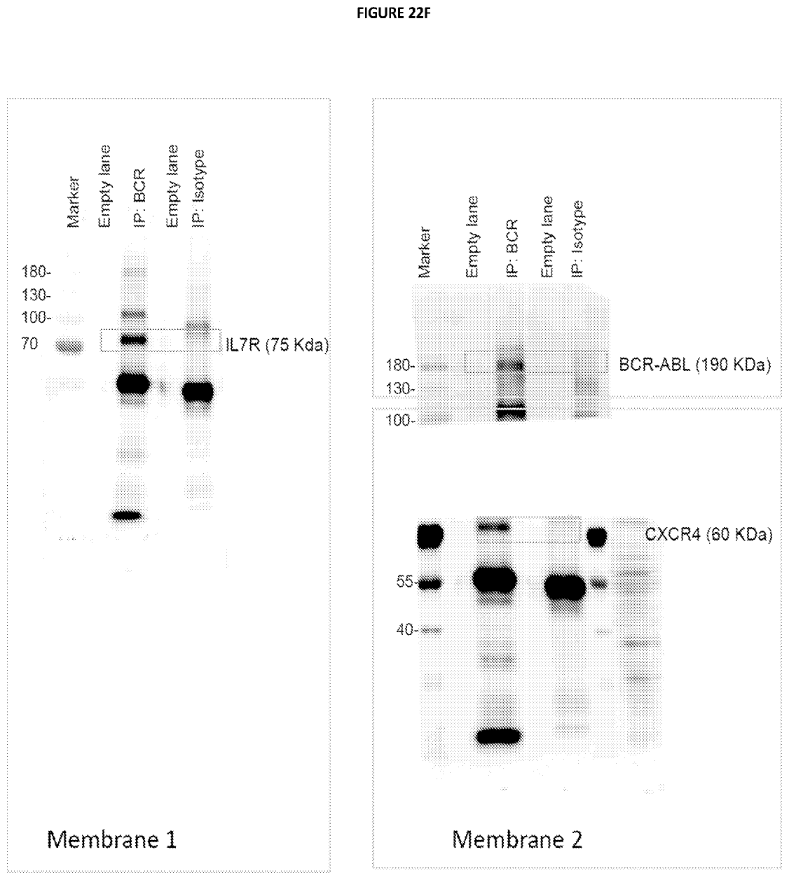

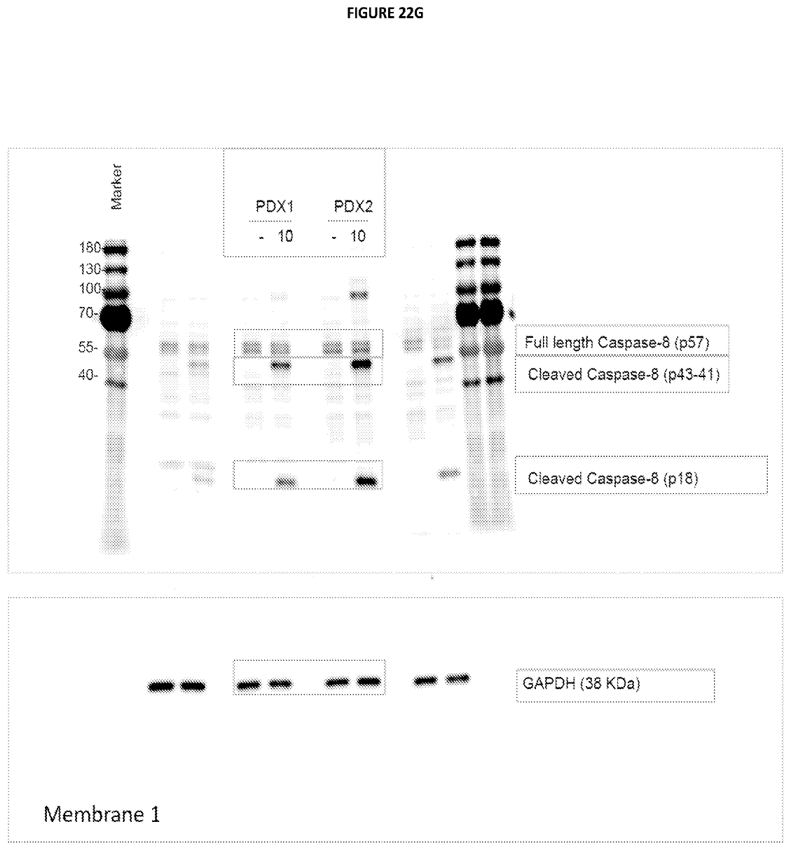

Since the above results show that IL7R is crucial for the transforming signals initiated by BCR-ABL1 in Ph+ ALL, we investigated whether inhibition of IL7R signaling using ruxolitinib, a JAK1/JAK2 kinase inhibitor, can interfere with the survival of BCR-ABL1 transformed cells or enhance the effect of kinase inhibitors on these cells. We found that treatment with ruxolitinib along with imatinib prevented the IL7-driven rescue of BCR-ABL1 transformed pre-B cells in vitro (FIG. 17a-c). To further study the consequences of ruxolitinib on leukemia cells in vivo, we injected human BCR-ABL⁺ ALL cells into NSG mice and monitored the recipient mice under imatinib, ruxolitinib, or combined imatinib/ruxolitinib treatment. We found that combination treatment was unable to prolong the survival of recipient mice or to reduce the percentage of leukemic cells in the BM and in the spleen (FIG. 17d-f) suggesting that, in contrast to the in vitro results, ruxolitinib cannot support imatinib in a xenograft model and therefore may not be suitable for ALL treatment in vivo. Therefore, we tested whether direct targeting of the IL7R using specific monoclonal antibodies may interfere with its function in leukemia. To this end, we injected imatinib-resistant BCR-ABL1⁺ ALL patient cells³⁵ into NSG mice and treated them with monoclonal antibody specific for human IL7Rα⁴ (FIG. 7b-d). As expected, imatinib was unable to prevent leukemia development and, therefore, the leukemia burden was increased and the survival of imatinib-treated mice was reduced similar to that of control mice (FIG. 7a-b). In contrast, anti-IL7R antibody significantly delayed leukemia onset in vivo and led to a significantly expanded survival time of the respective animals (FIG. 7a-b and FIG. 18a-b). Interestingly, the xenograft patient material showed an upregulation in BCR-ABL1 expression compared to human BCR-ABL1+ cell lines TOM-1 and SUP-B15 (FIG. 7c) which may explain the imatinib-resistant phenotype³⁵. Indeed, we found that regulated long-term exposure to imatinib can lead to upregulation of BCR-ABL1 expression and to imatinib-resistance in vitro (FIG. 19). The xenograft patient material which was used lacked the BCR-ABL1 gate keeper mutation, known as T3151 kinase domain mutation, which leads to resistance against ABL1 inhibitors^{36,37}. Therefore, we repeated the experiment using T3151-positive, imatinib resistant, xenograft patient material³⁸. Treatment of recipient mice with anti-IL7R antibody significantly delayed leukemia onset and led to significant prolongation of the survival time of the respective animals (FIG. 7d-f and FIG. 18c). As NSG mice lack NK cells, we excluded that it led to cell death via antibody-dependent cell-mediated cytotoxicity (ADCC) as previously described by other IL7R antibodies³⁹. Similarly, the antibody which we used does not block IL7 binding (FIG. 20a), alternatively, it disrupts the scaffold between IL7R and CXCR4 (Supplementary FIG. 20b). In addition, antibody treatment seems to enhance apoptosis as shown by increased cleavage of caspase-8⁴⁰ (FIG. 20c).

Together, these experiments show that IL7R plays a pivotal role in the survival of ALL and that targeting IL7R via specific antibodies exerts a profound effect on elimination of kinase inhibitor-resistant ALL in vivo.

I. Discussion

Previous studies demonstrated remarkable outcome improvements in Ph+ ALL patients upon imatinib integration into chemotherapy⁴¹. However, acquired drug resistance is still a crucial issue that leads to relapse of the disease and unfavorable outcome^{3,42}. A thorough understanding of the molecular mechanisms involved in BCR-ABL1-mediated transformation is required in order to provide therapeutic alternatives for Ph+ ALL patients, particularly those who developed TM-resistance. In this study, we employed several genetically modified systems as well as preclinical xenograft models to better understand BCR-ABL1-induced transformation.

Interestingly, our data show that BCR-ABL1 oncogene regulates the expression and function of the signaling pathways of IL7R and CXCR4 in a concerted manner. This combined regulation is important because both receptors are required for the growth and survival of BCR-ABL1-transformed pre-B cells. Importantly, IL7R and CXCR4 act in close proximity thereby allowing their downstream signaling pathways to synergize and enable BCR-ABL1-induced pre-B cell transformation. In this synergism, CXCR4 attracts the oncogenic kinase BCR-ABL1 while IL7R conveys the JAK/STAT signaling machinery. Importantly, this complex seems to act in a ligand-independent manner to activate multiple downstream signaling pathways and is required for the survival of mouse and human leukemia cells in both in vitro as well as in vivo preclinical xenograft model.

Our results indicate that BCR-ABL1 utilizes the IL7R signaling machinery for pre-B cell transformation and growth factor-independent proliferation and that the feedback regulation of this machinery is a crucial part of the transformation process. For instance, deregulated BCR-ABL1 kinase activity may result in uncontrolled STAT5 phosphorylation and negative feedback regulation of IL7R expression leading to cell death. Previous report suggested that BCR-ABL oncogene mimics pre-BCR signaling by activating STAT5 in one hand and repressing BCL6 expression on the other hand. STAT5 was also shown to directly downregulate BCL6 expression in response to IL7 stimulation⁴³. This is in agreement with our data showing that BCR-ABL transformation downregulates the transcription repressor BCL6. Thus, BCR-ABL1-mediated pre-B cell transformation requires an equilibrium between kinase activity and negative feedback regulation of IL7R signaling. In full agreement, BCR-ABL1 transformed pre-B cells require multiple phosphatases that are most likely involved in stabilizing this equilibrium³⁸ which can be targeted for efficient treatment of Ph+ ALL. It is feasible that additional players participate in regulating IL7R expression in ALL. For example, it was previously shown that Ikaros negatively regulates IL7R promoter and that Ikaros deficiency in ALL patients is correlated with increased IL7R expression⁴⁴. Similarly, the common IKZF1 deletion leading to dominant negative IK6 isoform⁴⁵ resulted in increased IL7R expression in BCR-ABL+ cells⁴⁶. Thus, co-occurring genomic alterations such as IKZF1 deletion remain to be addressed in future studies.

Previous reports showed that combined targeting of BCR-ABL1 and JAK2 using dasatinib and ruxolitinib, respectively, reduced leukemia engraftment and prolonged survival⁴⁷. However, these mice eventually relapsed and died from leukemia which suggest that ruxolitinib treatment is inefficient in vivo⁴⁷. This is in agreement with our results showing that inhibition of the kinases JAK1/JAK2 by ruxolitinib, applied either alone or in combination with imatinib, was not able to provide any therapeutic advantage for xenograft animal models injected with Ph+ ALL patient material. It is conceivable that reduced drug availability or insufficient inhibition of IL7R signaling, as ruxolitinib mainly inhibits JAK1 and JAK2 while IL7R can also activate JAK3, are responsible for the inability of ruxolitinib to block the development of Ph+ ALL in vivo.

Intriguingly, our experiments point to an unpredicted escape mechanism of transformed cells during TM treatment. Since leukemic cells maintain the expression of growth factor receptors such as IL7R, which is used as scaffold for organizing the oncogenic signaling machinery, the presence of IL7 in certain niches might provide the transformed cells with escape mechanisms upon treatment with inhibitors blocking BCR-ABL1⁴⁸. This scenario is also possible for other growth factor receptors and their respective cytokines. For example, it has been shown that IL3 can rescue BCR-ABL+ CML cells from cell death induced by BCR-ABL inhibitors^{47,49}. Although our data showed that several receptors were upregulated in response to imatinib (such as IL7R, CXCR4 and CRLF2), IL7 showed a unique potential to rescue the cells under kinase inhibitor treatment. Thus, it is conceivable that, during treatment of Ph+ ALL patients with inhibitors blocking BCR-ABL1 kinase activity, IL7R-driven survival pathways in ALL cells are activated in microenvironments containing IL7 thereby enabling the survival of ALL cells. Ph+ ALL cells that survive treatment with BCR-ABL inhibitors in microenvironments containing IL7 may act as leukemia initiating cells and disseminate to other locations when inhibitor concentrations decline or when inhibitor resistance is induced by somatic mutations. This scenario is further supported by the elevated amounts of IL7 detected in ALL patients^{9,10}. Thus, understanding the molecular mechanisms of BCR-ABL1-induced transformation is important for identifying TKI escape mechanisms and for developing strategies that prevent such escape.

Our findings may also have important therapeutic implications in other leukemia subtypes with similar gene expression such as Ph-like ALL. For example, at least 90% of patients with Ph-like ALL showed kinase-activating alterations (e.g., in ABL1/ABL2 and JAK2), sequence mutations in IL7R as well as an activation of phosphorylated STAT5⁵⁰. This suggests that IL7R might also be a potential therapeutic target for several BCP-ALL patients who are not Ph+ as wel1⁴. Nevertheless, additional work would be required to investigate whether our model also function in Ph-like ALL. Since IL7R expression and function is critical for proper lymphopoiesis, targeting this pathway may have effects on other normal cells. For instance, previous studies showed that mice deficient in IL7R showed depletion in both B and T lymphocytes⁵¹. In humans, mutations in the IL7Ra result in severe combined immunodeficiency (SCID) which is associated with absence of T cells and normal numbers, nevertheless inactive, B cells⁵². Accordingly, targeting IL7Ra using specific antibodies may also affect T cells⁵³ and lead to immunodeficiency in patients. However, a recent study showed that treating healthy subjects with anti-human IL7R antibody was well tolerated and did not result in obvious alterations in immune cell populations and inflammatory cytokine profiles⁵⁴. Thus, treatment with anti-IL7R antibodies might provide a key therapeutic approach especially for TM-resistant ALL once the different antibodies are characterized regarding their side-effects and compared with standard chemotherapy in appropriate clinical trials.

### REFERENCES

1. Ribeiro, R. C., et al. Clinical and biologic hallmarks of the Philadelphia chromosome in childhood acute lymphoblastic leukemia. Blood 70, 948-953 (1987).
2. Arico, M., et al. Outcome of treatment in children with Philadelphia chromosome-positive acute lymphoblastic leukemia. N Engl J Med 342, 998-1006 (2000).
3. Schultz, K. R., et al. Long-term follow-up of imatinib in pediatric Philadelphia chromosome-positive acute lymphoblastic leukemia: Children's Oncology Group study AALL0031. Leukemia 28, 1467-1471 (2014).
4. Alsadeq, A., et al. IL7R is associated with CNS infiltration and relapse in pediatric B-cell precursor acute lymphoblastic leukemia. Blood (2018).
5. Mazzucchelli, R. & Durum, S. K. Interleukin-7 receptor expression: intelligent design. Nat Rev Immunol 7, 144-154 (2007).
6. Park, L. S., et al. Cloning of the murine thymic stromal lymphopoietin (TSLP) receptor: Formation of a functional heteromeric complex requires interleukin 7 receptor. The Journal of experimental medicine 192, 659-670 (2000).
7. Geng, H., et al. Self-enforcing feedback activation between BCL6 and pre-B cell receptor signaling defines a distinct subtype of acute lymphoblastic leukemia. Cancer cell 27, 409-425 (2015).
8. Hertzberg, L., et al. Down syndrome acute lymphoblastic leukemia, a highly heterogeneous disease in which aberrant expression of CRLF2 is associated with mutated JAK2: a report from the International BFM Study Group. Blood 115, 1006-1017 (2010).
9. Sasson, S. C., et al. IL-7 receptor is expressed on adult pre-B-cell acute lymphoblastic leukemia and other B-cell derived neoplasms and correlates with expression of proliferation and survival markers. Cytokine 50, 58-68 (2010).
10. Mullighan, C. G. The molecular genetic makeup of acute lymphoblastic leukemia. Hematology/the Education Program of the American Society of Hematology. American Society of Hematology. Education Program 2012, 389-396 (2012).
11. Shochat, C., et al. Gain-of-function mutations in interleukin-7 receptor-alpha (IL7R) in childhood acute lymphoblastic leukemias. The Journal of experimental medicine 208, 901-908 (2011).
12. Kerdiles, Y. M., et al. Foxol links homing and survival of naive T cells by regulating L-selectin, CCR7 and interleukin 7 receptor. Nat Immunol 10, 176-184 (2009).
13. Herzog, S., Reth, M. & Jumaa, H. Regulation of B-cell proliferation and differentiation by pre-B-cell receptor signalling. Nat Rev Immunol 9, 195-205 (2009).
14. Herzog, S., et al. SLP-65 regulates immunoglobulin light chain gene recombination through the P1(3)K-PKB-Foxo pathway. Nat Immunol 9, 623-631 (2008).
15. Guo, F., et al. CXCL12/CXCR4: a symbiotic bridge linking cancer cells and their stromal neighbors in oncogenic communication networks. Oncogene 35, 816-826 (2016).
16. van den Berk, L. C., et al. Disturbed CXCR4/CXCL12 axis in paediatric precursor B-cell acute lymphoblastic leukaemia. Br J Haematol 166, 240-249 (2014).
17. Hayashi, S., et al. Stepwise progression of B lineage differentiation supported by interleukin 7 and other stromal cell molecules. The Journal of experimental medicine 171, 1683-1695 (1990).
18. Nagasawa, T. CXCL12/SDF-1 and CXCR4. Front Immunol 6, 301 (2015).
19. Haferlach, T., et al. Clinical utility of microarray-based gene expression profiling in the diagnosis and subclassification of leukemia: report from the International Microarray Innovations in Leukemia Study Group. J Clin Oncol 28, 2529-2537 (2010).
20. Li, J. F., et al. Transcriptional landscape of B cell precursor acute lymphoblastic leukemia based on an international study of 1,223 cases. Proc Natl Acad Sci USA 115, E11711-E11720 (2018).
21. Jacobs, S. R., Michalek, R. D. & Rathmell, J. C. IL-7 is essential for homeostatic control of T cell metabolism in vivo. J Immunol 184, 3461-3469 (2010).
22. Peschon, J. J., et al. Early lymphocyte expansion is severely impaired in interleukin 7 receptor-deficient mice. The Journal of experimental medicine 180, 1955-1960 (1994).
23. Nie, Y., et al. The role of CXCR4 in maintaining peripheral B cell compartments and humoral immunity. The Journal of experimental medicine 200, 1145-1156 (2004).
24. Ptasznik, A., et al. Crosstalk between BCR/ABL oncoprotein and CXCR4 signaling through a Src family kinase in human leukemia cells. The Journal of experimental medicine 196, 667-678 (2002).
25. Becker, M., Hobeika, E., Jumaa, H., Reth, M. & Maity, P. C. CXCR4 signaling and function require the expression of the IgD-class B-cell antigen receptor. Proc Natl Acad Sci USA 114, 5231-5236 (2017).
26. Shah, N. P., et al. Overriding imatinib resistance with a novel ABL kinase inhibitor. Science 305, 399-401 (2004).
27. Flemming, A., Brummer, T., Reth, M. & Jumaa, H. The adaptor protein SLP-65 acts as a tumor suppressor that limits pre-B cell expansion. Nat Immunol 4, 38-43 (2003).
28. Moriguchi, M., et al. CXCL12 signaling is independent of Jak2 and Jak3. J Biol Chem 280, 17408-17414 (2005).
29. Nosaka, T. & Kitamura, T. Janus kinases (JAKs) and signal transducers and activators of transcription (STATs) in hematopoietic cells. Int J Hematol 71, 309-319 (2000).
30. Goetz, C A., Harmon, I. R., O'Neil, J. J., Burchill, M. A. & Farrar, M. A. STAT5 activation underlies IL7 receptor-dependent B cell development. J Immunol 172, 4770-4778 (2004).
31. Dominguez-Sola, D., et al. The FOXO1 Transcription Factor Instructs the Germinal Center Dark Zone Program. Immunity 43, 1064-1074 (2015).
32. Sander, S., et al. P13 Kinase and FOXO1 Transcription Factor Activity Differentially Control B Cells in the Germinal Center Light and Dark Zones. Immunity 43, 1075-1086 (2015).
33. Santos, S. C., et al. Constitutively active STAT5 variants induce growth and survival of hematopoietic cells through a PI 3-kinase/Akt dependent pathway. Oncogene 20, 2080-2090 (2001).
34. Onishi, M., et al. Identification and characterization of a constitutively active STAT5 mutant that promotes cell proliferation. Mol Cell Biol 18, 3871-3879 (1998).
35. Frismantas, V., et al. Ex vivo drug response profiling detects recurrent sensitivity patterns in drug-resistant acute lymphoblastic leukemia. Blood 129, e26-e37 (2017).
36. Baccarani, M., et al. Chronic myeloid leukemia: an update of concepts and management recommendations of European LeukemiaNet. J Clin Oncol 27, 6041-6051 (2009).
37. Bradeen, H. A., et al. Comparison of imatinib mesylate, dasatinib (BMS-354825), and nilotinib (AMN107) in an N-ethyl-N-nitrosourea (ENU)-based mutagenesis screen: high efficacy of drug combinations. Blood 108, 2332-2338 (2006).
38.
39. Hixon, J. A., et al. New anti-IL-7Ralpha monoclonal antibodies show efficacy against T cell acute lymphoblastic leukemia in pre-clinical models. Leukemia (2019).
40. Tummers, B. & Green, D. R. Caspase-8: regulating life and death. Immunol Rev 277, 76-89 (2017).
41. Biondi, A., et al. Imatinib after induction for treatment of children and adolescents with Philadelphia-chromosome-positive acute lymphoblastic leukaemia (EsPhALL): a randomised, open-label, intergroup study. Lancet Oncol 13, 936-945 (2012).
42. Druker, B. J., et al. Five-year follow-up of patients receiving imatinib for chronic myeloid leukemia. N Engl J Med 355, 2408-2417 (2006).
43. Ribeiro, D., et al. STAT5 is essential for IL-7-mediated viability, growth, and proliferation of T-cell acute lymphoblastic leukemia cells. Blood Adv 2, 2199-2213 (2018).
44. Ge, Z., et al. Co-existence of IL7R high and SH2B3 low expression distinguishes a novel high-risk acute lymphoblastic leukemia with Ikaros dysfunction. Oncotarget 7, 46014-46027 (2016).
45. Mullighan, C. G., et al. BCR-ABL1 lymphoblastic leukaemia is characterized by the deletion of Ikaros. Nature 453, 110-114 (2008).
46. Churchman, M. L., et al. Efficacy of Retinoids in IKZF1-Mutated BCR-ABL1 Acute Lymphoblastic Leukemia. Cancer cell 28, 343-356 (2015).
47. Appelmann, I., et al. Janus kinase inhibition by ruxolitinib extends dasatinib- and dexamethasone-induced remissions in a mouse model of Ph+ ALL. Blood 125, 1444-1451 (2015).
48. Singh, H., et al. A screening-based approach to circumvent tumor microenvironment-driven intrinsic resistance to BCR-ABL+ inhibitors in Ph+ acute lymphoblastic leukemia. J Biomol Screen 19, 158-167 (2014).
49. Dorsey, J. F., et al. Interleukin-3 protects Bcr-Abl-transformed hematopoietic progenitor cells from apoptosis induced by Bcr-Abl tyrosine kinase inhibitors. Leukemia 16, 1589-1595 (2002).
50. Roberts, K. G., et al. Targetable kinase-activating lesions in Ph-like acute lymphoblastic leukemia. N Engl J Med 371, 1005-1015 (2014).
51. von Freeden-Jeffry, U., et al. Lymphopenia in interleukin (IL)-7 gene-deleted mice identifies IL-7 as a nonredundant cytokine. The Journal of experimental medicine 181, 1519-1526 (1995).
52. Puel, A., Ziegler, S. F., Buckley, R. H. & Leonard, W. J. Defective IL7R expression in T(−) B(+)NK(+) severe combined immunodeficiency. Nature genetics 20, 394-397 (1998).
53. Belarif, L., et al. IL-7 receptor blockade blunts antigen-specific memory T cell responses and chronic inflammation in primates. Nat Commun 9, 4483 (2018).
54. Ellis, J., et al. Anti-IL-7 receptor alpha monoclonal antibody (GSK2618960) in healthy subjects—a randomized, double-blind, placebo-controlled study. Br J Clin Pharmacol 85, 304-315 (2019).
55. Huber, W., et al. Orchestrating high-throughput genomic analysis with Bioconductor. Nat Methods 12, 115-121 (2015).
56. R Core Team. R: A language and environment for statistical computing. (R Foundation for Statistical Computing, Vienna, Austria. , 2015).
57. Subramanian, A., et al. Gene set enrichment analysis: a knowledge-based approach for interpreting genome-wide expression profiles. Proc Natl Acad Sci USA 102, 15545-15550 (2005).
58. Levit-Zerdoun, E., et al. Survival of Igalpha-Deficient Mature B Cells Requires BAFF-R Function. J Immunol 196, 2348-2360 (2016).
59. Krause, S., et al. Mer tyrosine kinase promotes the survival of t(1;19)-positive acute lymphoblastic leukemia (ALL) in the central nervous system (CNS). Blood 125, 820-830 (2015).
60. Alsadeq, A., et al. Effects of p38alpha/beta inhibition on acute lymphoblastic leukemia proliferation and survival in vivo. Leukemia (2015).
61. Alsadeq, A., et al. The role of ZAP70 kinase in acute lymphoblastic leukemia infiltration into the central nervous system. Haematologica 102, 346-355 (2017).
62. Kohler, F., et al. Autoreactive B cell receptors mimic autonomous pre-B cell receptor signaling and induce proliferation of early B cells. Immunity 29, 912-921 (2008).

What is claimed is:

1. A composition comprising an antibody that specifically binds to interleukin 7 receptor (IL7R), wherein the antibody does not prevent binding of IL7R to interleukin 7 (IL7).

2. The composition of claim 1, wherein the antibody does not bind to a ligand binding site of IL7R.

3. The composition of claim 1 or 2, wherein the antibody binds to a ligand binding site of IL7R.

4. A composition comprising an antibody that specifically binds to interleukin 7 receptor (IL7R), wherein the antibody comprises:
a variable heavy domain (VH) comprising vhCDRs 1-3 having amino acid sequences of SEQ ID NOs:2-4, respectively; and
a variable light domain (VL) comprising vlCDRs 1-3 having amino acid sequences of SEQ ID NOs:9-11, respectively.

5. A composition comprising an antibody that specifically binds to interleukin 7 receptor (IL7R), wherein the antibody comprises:
a variable heavy domain (VH) from a heavy chain having an amino acid sequence of SEQ ID NO:1; and
a variable light domain (VL) from a light chain having an amino acid sequence of SEQ ID NO:8.

6. The composition of claim 5, wherein the antibody comprises:
a heavy chain having an amino acid sequence of SEQ ID NO:1; and
a light chain having an amino acid sequence of SEQ ID NO:8.

7. A composition comprising an antibody that specifically binds to interleukin 7 receptor (IL7R), wherein the antibody comprises:
a variable heavy domain (VH) comprising vhCDRs 1-3 having amino acid sequences of SEQ ID NOs:2, 6, and 4, respectively;
a variable light domain (VL) comprising vlCDRs 1-3 having amino acid sequences of SEQ ID NOs:9-11, respectively.

8. A composition comprising an antibody that specifically binds to interleukin 7 receptor (IL7R), wherein the antibody comprises:
a variable heavy domain (VH) from a heavy chain having an amino acid sequence of SEQ ID NO:5;
a variable light domain (VL) from a light chain having an amino acid sequence of SEQ ID NO:8.

9. The composition of claim 8, wherein the antibody comprises:
a heavy chain having an amino acid sequence of SEQ ID NO:5; and
a light chain having an amino acid sequence of SEQ ID NO:8.

10. A composition comprising an antibody that specifically binds to interleukin 7 receptor (IL7R), wherein the antibody comprises:
a variable heavy domain (VH) from a heavy chain having an amino acid sequence of SEQ ID NO:7;
a variable light domain (VL) from a light chain having an amino acid sequence of SEQ ID NO:8.

11. The composition of claim 10, wherein the antibody comprises:
a heavy chain having an amino acid sequence of SEQ ID NO:7; and
a light chain having an amino acid sequence of SEQ ID NO:8.

12. A composition comprising an antibody that competes with the antibody of any one of claims 1 to 11 for binding to interleukin 7 receptor (IL7R).

13. A composition comprising:
a first nucleic acid comprising a first polynucleotide sequence encoding the variable heavy domain (VH) of the antibody of any one of claims 1 to 12, and
a second nucleic acid comprising a second polynucleotide sequence encoding the variable light domain (VL) of the same antibody.

14. An expression vector comprising the first and second nucleic acids of claim 13.

15. A host cell comprising the expression vector of claim 14.

16. A method of making the antibody of any one of claims 1 to 12 comprising
a) culturing the host cell of claim 14 under conditions wherein the antibody is produced; and
b) recovering the antibody.

17. The method of claim 16, further comprising humanizing the antibody.

18. A method of treating acute leukemia in a patient in need comprising administering an antibody that specifically binds to interleukin 7 receptor (IL7R) to the patient.

19. The method of claim 18, wherein the antibody that specifically binds to interleukin 7 receptor (IL7R) is the antibody of any one of claims 1 to 12.

20. The method of claim 18 or 19, wherein the antibody is used as a monotherapy.

21. The method of claim 18 or 19, wherein the antibody is used in combination with a standard chemotherapy.

22. The method of claim 21, wherein the antibody is administered prior to, simultaneously with, or subsequent to the administration of one or more chemotherapeutic agents.

23. The method of claim 18 or 19, wherein the antibody is used in combination with a kinase inhibitor (such as imatinib).

24. The method of claim 23, wherein the antibody is administered prior to, simultaneously with, or subsequent to the administration of the kinase inhibitor (such as imatinib).

25. The method of any one of claims 18 to 24, wherein prior to the treatment the patient has relapsed following TM therapy.

26. The method of any one of claims 18 to 25, wherein the patient has circulating malignant cells.

27. The method of any one of claims 18 to 26, wherein circulating malignant cells in the patient expresses IL7R.

28. The method of any one of claims 18 to 27, wherein the leukemia is resistant leukemia.

29. The method of any one of claims 18 to 28, wherein the leukemia is acute lymphoblastic leukemia (ALL).

30. The method of claim 29, wherein the ALL is Philadelphia chromosome positive acute lymphoblastic leukemia (Ph+ALL).

31. The method of claim 29, wherein the ALL is resistant ALL.

32. The method of claim 29, wherein the ALL is pediatric ALL.

33. The antibody of any one of claims 1 to 13 for use in treating leukemia (including resistant leukemia).

34. The antibody of any one of claims 1 to 13 for use in treating ALL.

35. The antibody of any one of claims 1 to 13 for use in treating Philadelphia chromosome positive acute lymphoblastic leukemia (Ph+ALL).

36. The antibody of any one of claims 1 to 13 for use in treating resistant ALL.

37. The antibody of any one of claims 1 to 13 for use in treating pediatric ALL.

38. A method of treating a patient with Ph+ALL by administering an anti-IL7R antibody that does not compete for binding with IL7.


SEQUENCE LISTING (15 sequences)

SEQ ID NO: 1 - protein, 118 aa, Artificial Sequence
  Synthetic variable heavy chain
QVQLKESGPGLVAPSQSLSITCTVSGFSLTSYGVHWVRQPPGKGLEWLGVIWTGGSTHYNSALMSRLSISKDNSKSQVFLKMNSLQTDDTAMYYCAREGDYYASFAYWGQGTLVTVSA

SEQ ID NO: 2 - protein, 8 aa, Artificial Sequence
  Synthetic variable heavy chain CDR1
GFSLTSYG

SEQ ID NO: 3 - protein, 7 aa, Artificial Sequence
  Synthetic variable heavy chain CDR2
IWTGGST

SEQ ID NO: 4 - protein, 12 aa, Artificial Sequence
  Synthetic variable heavy chain CDR3
AREGDYYASFAY

SEQ ID NO: 5 - protein, 118 aa, Artificial Sequence
  Synthetic variable heavy chain
QVQLKESGPGLVAPSQSLSITCTVSGFSLTSYGVHWVRQPPGKGLEWLGVIWAGGSTHYNSALMSRLSISKDNSKSQVFLKMNSLQTDDTAMYYCAREGDYYASFAYWGQGTLVTVSA

SEQ ID NO: 6 - protein, 7 aa, Artificial Sequence
  Synthetic variable heavy chain CDR2
IWTGGST

SEQ ID NO: 7 - protein, 118 aa, Artificial Sequence
  Synthetic variable heavy chain
QVQLKESGPGLVAPSQSLSITCTVSGFSLTSYGVHWVRQPPGKGLEWLGVIWTGGSTNYNSALMSRLSISKDNSKSQVFLKMNSLQTDDTAMYYCAREGDYYASFAYWGQGTLVTVSA

SEQ ID NO: 8 - protein, 108 aa, Artificial Sequence
  Synthetic variable light chain
DIQMNQSPSSLSASLGDTITITCHASQNINVWLSWYQQKPGNIPKLLIYKASNLHTGVPSRFSGSGSGTGFTLTISSLQPEDIATYYCQQGQSYPYTFGGGTKLEIKR

SEQ ID NO: 9 - protein, 6 aa, Artificial Sequence
  Synthetic variable light chain CDR1
QNINVW

SEQ ID NO: 10 - protein, 3 aa, Artificial Sequence
  Synthetic variable light chain CDR2
KAS

SEQ ID NO: 11 - protein, 9 aa, Artificial Sequence
  Synthetic variable light chain CDR3
QQGQSYPYT

SEQ ID NO: 12 - protein, 330 aa, Artificial Sequence [sequence omitted]

SEQ ID NO: 13 - protein, 326 aa, Artificial Sequence
  Synthetic protein - IgG2 associated sequence
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 14 - protein, 377 aa, Artificial Sequence
  Synthetic protein - IgG3 associated sequence
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 15 - protein, 327 aa, Artificial Sequence
  Synthetic protein - IgG4 associated sequence
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK